SEQUENCE LISTING (86 sequences)

SEQ ID NO: 1 - protein, 385 aa, synthetic construct
  REGION 1..385: note = Description of Artificial Sequence: Syntheticpolypeptide
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 2 - protein, 686 aa, synthetic construct
  REGION 1..686: note = Description of Artificial Sequence: Syntheticpolypeptide
APPKSVIRWCTISSPEEKKCNNLRDLTQQERISLTCVQKATYLDCIKAIANNEADAISLDGGQVFEAGLAPYKLKPIAAEIYEHTEGSTTSYYAVAVVKKGTEFTVNDLQGKNSCHTGLGRSAGWNIPIGTLLHWGAIEWEGIESGSVEQAVAKFFSASCVPGATIEQKLCRQCKGDPKTKCARNAPYSGYSGAFHCLKDGKGDVAFVKHTTVNENAPDLNDEYELLCLDGSRQPVDNYKTCNWARVAAHAVVARDDNKVEDIWSFLSKAQSDFGVDTKSDFHLFGPPGKKDPVLKDFLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISNLKTCNPSDILQMCSFLEGK

SEQ ID NO: 3 - protein, 186 aa, synthetic construct
  REGION 1..186: note = Description of Artificial Sequence: Syntheticpolypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 4 - protein, 186 aa, synthetic construct
  REGION 1..186: note = Description of Artificial Sequence: Syntheticpolypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 5 - protein, 426 aa, synthetic construct
  REGION 1..426: note = Description of Artificial Sequence: Syntheticpolypeptide
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 6 - protein, 388 aa, synthetic construct
  REGION 1..388: note = Description of Artificial Sequence: Syntheticpolypeptide
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 7 - protein, 2087 aa, synthetic construct
  REGION 1..2087: note = Description of Artificial Sequence: Syntheticpolypeptide
KEPVQIVQVSTVGRSECTTWGNFHFHTFDHVKFTFPGTCTYVFASHCNDSYQDFNIKIRRSDKNSHLIYFTVTTDGVILEVKETGITVNGNQIPLPFSLKSILIEDTCAYFQVTSKLGLTLKWNWADTLLLDLEETYKEKICGLCGNYDGNKKNDLILDGYKMHPRQFGNFHKVEDPSEKCPDVRPDDHTGRHPTEDDNRCSKYKKMCKKLLSRFGNCPKVVAFDDYVATCTEDMCNCVVNSSHSDLVSSCICSTLNQYSRDCVLSKGDPGEWRTKELCYQECPSNMEYMECGNSCADTCADPERSKICKAPCTDGCFCPPGTILDDLGGKKCVPRDSCPCMFQGKVYSSGGTYSTPCQNCTCKGGHWSCTSLPCSGSCSIDGGFHITTFDNKKFNFHGNCHYVLAKNTDDTFVVIGEIIQCGTSKTMTCLKNVLVTLGRTTIKICSCGSIYMNNFIVKLPVSKDGITIFRPSTFFIKILSSTGVQIRVQMKPVMQLSITVDHSYQNRTSGLCGNFNNIQTDDFRTATGAVEDSAAAFGNSWKTRASCFDVEDSFEDPCSNSVDKEKFAQHWCALLSNISSTFAACHSVVDPSVYIKRCMYDTCNAEKSEVALCSVLSTYSRDCAAAGMTLKGWRQGICDPSEECPETMVYNYSVKYCNQSCRSLDEPDPLCKVQIAPMEGCGCPEGTYLNDEEECVTPDDCPCYYKGKIVQPGNSFQEDKLLCKCIQGRLDCIGETVLVKDCPAPMYYFNCSSAGPGAIGSECQKSCKTQDMHCYVTECVSGCMCPDGLVLDGSGGCIPKDQCPCVHGGHFYKPGETIRVDCNTCTCNKRQWNCTDSPCKGTCTVYGNGHYMSFDGEKFDFLGDCDYILAQDFCPNNMDAGTFRIVIQNNACGKSLSICSLKITLIFESSEIRLLEGRIQEIATDPGAEKNYKVDLRGGYIVIETTQGMSFMWDQKTTVVVHVTPSFQGKVCGLCGDFDGRSRNDFTTRGQSVEMSIQEFGNSWKITSTCSNINMTDLCADQPFKSALGQKHCSIIKSSVFEACHSKVNPIPYYESCVSDFCGCDSVGDCECFCTSVAAYARSCSTAGVCINWRTPAICPVFCDYYNPPDKHEWFYKPCGAPCLKTCRNPQGKCGNILYSLEGCYPECSPDKPYFDEERRECVSLPDCTSCNPEEKLCTEDSKDCLCCYNGKTYPLNETIYSQTEGTKCGNAFCGPNGMIIETFIPCSTLSVPAQEQLMQPVTSAPLLSTEATPCFCTDNGQLIQMGENVSLPMNISGHCAYSICNASCQIELIWAECKVVQTEALETCEPNSEACPPTAAPNATSLVPATALAPMSDCLGLIPPRKFNESWDFGNCQIATCLGEENNIKLSSITCPPQQLKLCVNGFPFMKHHDETGCCEVFECQCICSGWGNEHYVTFDGTYYHFKENCTYVLVELIQPSSEKFWIHIDNYYCGAADGAICSMSLLIFHSNSLVILTQAKEHGKGTNLVLFNDKKVVPDISKNGIRITSSGLYIIVEIPELEVYVSYSRLAFYIKLPFGKYYNNTMGLCGTCTNQKSDDARKRNGEVTDSFKEMALDWKAPVSTNRYCNPGISEPVKIENYQHCEPSELCKIIWNLTECHRVVPPQPYYEACVASRCSQQHPSTECQSMQTYAALCGLHGICVDWRGQTNGQCEATCARDQVYKPCGEAKRNTCFSREVIVDTLLSRNNTPVFVEGCYCPDGNILLNEHDGICVSVCGCTAQDGSVKKPREAWEHDCQYCTCDEETLNISCFPRPCAKSPPINCTKEGFVRKIKPRLDDPCCTETVCECDIKTCIINKTACDLGFQPVVAISEDGCCPIFSCIPKGVCVSEGVEFKPGAVVPKSSCEDCVCTDEQDAVTGTNRIQCVPVKCQTTCQQGFRYVEKEGQCCSQCQQVACVANFPFGSVTIEVGKSYKAPYDNCTQYTCTESGGQFSLTSTVKVCLPFEESNCVPGTVDVTSDGCCKTCIDLPHKCKRSMKEQYIVHKHCKSAAPVPVPFCEGTCSTYSVYSFENNEMEHKCICCHEKKSHVEKVELVCSEHKTLKFSYVHVDECGCVETKCPMRRT

SEQ ID NO: 8 - protein, 1185 aa, synthetic construct
  REGION 1..1185: note = Description of Artificial Sequence: Syntheticpolypeptide
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 9 - protein, 129 aa, synthetic construct
  REGION 1..129: note = Description of Artificial Sequence: Syntheticpolypeptide
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 10 - protein, 449 aa, synthetic construct
  REGION 1..449: note = Description of Artificial Sequence: Syntheticpolypeptide
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 11 - protein, 139 aa, synthetic construct
  REGION 1..139: note = Description of Artificial Sequence: Syntheticpolypeptide
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 12 - protein, 402 aa, synthetic construct
  REGION 1..402: note = Description of Artificial Sequence: Syntheticpolypeptide
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 13 - protein, 388 aa, synthetic construct; identical to SEQ ID NO: 6
  REGION 1..388: note = Description of Artificial Sequence: Syntheticpolypeptide

SEQ ID NO: 14 - protein, 18 aa, unidentified
  REGION 1..18: note = Description of Unknown:signal peptide
MQVKSIVNLLLACSLAVA

SEQ ID NO: 15 - protein, 22 aa, unidentified
  REGION 1..22: note = Description of Unknown:signal peptide
MQFNWNIKTVASILSALTLAQA

SEQ ID NO: 16 - protein, 17 aa, unidentified
  REGION 1..17: note = Description of Unknown:signal peptide
MYRNLIIATALTCGAYS

SEQ ID NO: 17 - protein, 16 aa, unidentified
  REGION 1..16: note = Description of Unknown:signal peptide
MNLYLITLLFASLCSA

SEQ ID NO: 18 - protein, 25 aa, unidentified
  REGION 1..25: note = Description of Unknown:signal peptide
MFEKSKFVVSFLLLLQLFCVLGVHG

SEQ ID NO: 19 - protein, 21 aa, unidentified
  REGION 1..21: note = Description of Unknown:signal peptide
MQFNSVVISQLLLTLASVSMG

SEQ ID NO: 20 - protein, 16 aa, unidentified
  REGION 1..16: note = Description of Unknown:signal peptide
MKSQLIFMALASLVAS

SEQ ID NO: 21 - protein, 19 aa, unidentified
  REGION 1..19: note = Description of Unknown:signal peptide
MKFAISTLLIILQAAAVFA

SEQ ID NO: 22 - protein, 23 aa, unidentified
  REGION 1..23: note = Description of Unknown:signal peptide
MKLLNFLLSFVTLFGLLSGSVFA

SEQ ID NO: 23 - protein, 20 aa, unidentified
  REGION 1..20: note = Description of Unknown:signal peptide
MIFNLKTLAAVAISISQVSA

SEQ ID NO: 24 - protein, 19 aa, unidentified
  REGION 1..19: note = Description of Unknown:signal peptide
MKISALTACAVTLAGLAIA

SEQ ID NO: 25 - protein, 18 aa, unidentified
  REGION 1..18: note = Description of Unknown:signal peptide
MSYLKISALLSVLSVALA

SEQ ID NO: 26 - protein, 20 aa, unidentified
  REGION 1..20: note = Description of Unknown:signal peptide
MLSTILNIFILLLFIQASLQ

SEQ ID NO: 27 - protein, 20 aa, unidentified
  REGION 1..20: note = Description of Unknown:signal peptide
MKLSTNLILAIAAASAVVSA

SEQ ID NO: 28 - protein, 19 aa, unidentified
  REGION 1..19: note = Description of Unknown:signal peptide
MFKSLCMLIGSCLLSSVLA

SEQ ID NO: 29 - protein, 19 aa, unidentified
  REGION 1..19: note = Description of Unknown:signal peptide
MKLAALSTIALTILPVALA

SEQ ID NO: 30 - protein, 23 aa, unidentified
  REGION 1..23: note = Description of Unknown:signal peptide
MSFSSNVPQLFLLLVLLTNIVSG

SEQ ID NO: 31 - protein, 18 aa, unidentified
  REGION 1..18: note = Description of Unknown:signal peptide
MQLQYLAVLCALLLNVQS

SEQ ID NO: 32 - protein, 19 aa, unidentified
  REGION 1..19: note = Description of Unknown:signal peptide
MKIHSLLLWNLFFIPSILG

SEQ ID NO: 33 - protein, 19 aa, unidentified
  REGION 1..19: note = Description of Unknown:signal peptide
MSTLTLLAVLLSLQNSALA

SEQ ID NO: 34 - protein, 20 aa, unidentified
  REGION 1..20: note = Description of Unknown:signal peptide
MINLNSFLILTVTLLSPALA

SEQ ID NO: 35 - protein, 16 aa, unidentified
  REGION 1..16: note = Description of Unknown:signal peptide
MFSLAVGALLLTQAFG

SEQ ID NO: 36 - protein, 16 aa, unidentified
  REGION 1..16: note = Description of Unknown:signal peptide
MKILSALLLLFTLAFA

SEQ ID NO: 37 - protein, 20 aa, unidentified
  REGION 1..20: note = Description of Unknown:signal peptide
MKVSTTKFLAVFLLVRLVCA

SEQ ID NO: 38 - protein, 19 aa, unidentified
  REGION 1..19: note = Description of Unknown:signal peptide
MQFGKVLFAISALAVTALG

SEQ ID NO: 39 - protein, 18 aa, unidentified
  REGION 1..18: note = Description of Unknown:signal peptide
MWSLFISGLLIFYPLVLG

SEQ ID NO: 40 - protein, 21 aa, unidentified
  REGION 1..21: note = Description of Unknown:signal peptide
MRNHLNDLVVLFLLLTVAAQA

SEQ ID NO: 41 - protein, 18 aa, unidentified
  REGION 1..18: note = Description of Unknown:signal peptide
MFLKSLLSFASILTLCKA

SEQ ID NO: 42 - protein, 20 aa, unidentified
  REGION 1..20: note = Description of Unknown:signal peptide
MFVFEPVLLAVLVASTCVTA

SEQ ID NO: 43 - protein, 22 aa, unidentified
  REGION 1..22: note = Description of Unknown:signal peptide
MVSLRSIFTSSILAAGLTRAHG

SEQ ID NO: 44 - protein, 22 aa, unidentified
  REGION 1..22: note = Description of Unknown:signal peptide
MFSPILSLEIILALATLQSVFA

SEQ ID NO: 45 - protein, 16 aa, unidentified
  REGION 1..16: note = Description of Unknown:signal peptide
MIINHLVLTALSIALA

SEQ ID NO: 46 - protein, 19 aa, unidentified
  REGION 1..19: note = Description of Unknown:signal peptide
MLALVRISTLLLLALTASA

SEQ ID NO: 47 - protein, 17 aa, unidentified
  REGION 1..17: note = Description of Unknown:signal peptide
MRPVLSLLLLLASSVLA

SEQ ID NO: 48 - protein, 24 aa, unidentified
  REGION 1..24: note = Description of Unknown:signal peptide
MVLIQNFLPLFAYTLFFNQRAALA

SEQ ID NO: 49 - protein, 21 aa, unidentified
  REGION 1..21: note = Description of Unknown:signal peptide
MKFPVPLLFLLQLFFIIATQG

SEQ ID NO: 50 - protein, 20 aa, unidentified
  REGION 1..20: note = Description of Unknown:signal peptide
MVSLTRLLITGIATALQVNA

SEQ ID NO: 51 - protein, 24 aa, unidentified
  REGION 1..24: note = Description of Unknown:signal peptide
MIFDGTTMSIAIGLLSTLGIGAEA

SEQ ID NO: 52 - protein, 31 aa, unidentified
  REGION 1..31: note = Description of Unknown:signal peptide
MVLVGLLTRLVPLVLLAGTVLLLVFVVLSGG

SEQ ID NO: 53 - protein, 16 aa, unidentified
  REGION 1..16: note = Description of Unknown:signal peptide
MLSILSALTLLGLSCA

SEQ ID NO: 54 - protein, 20 aa, unidentified
  REGION 1..20: note = Description of Unknown:signal peptide
MRLLHISLLSIISVLTKANA

SEQ ID NO: 55 - protein, 19 aa, unidentified
  REGION 1..19: note = Description of Unknown:signal peptide
MRFPSIFTAVLFAASSALA

SEQ ID NO: 56 - protein, 17 aa, unidentified
  REGION 1..17: note = Description of Unknown:signal peptide
MFKSVVYSILAASLANA

SEQ ID NO: 57 - protein, 19 aa, unidentified
  REGION 1..19: note = Description of Unknown:signal peptide
MLLQAFLFLLAGFAAKISA

SEQ ID NO: 58 - protein, 21 aa, unidentified
  REGION 1..21: note = Description of Unknown:signal peptide
MASSNLLSLALFLVLLTHANS

SEQ ID NO: 59 - protein, 16 aa, unidentified
  REGION 1..16: note = Description of Unknown:signal peptide
MNIFYIFLFLLSFVQG

SEQ ID NO: 60 - protein, 15 aa, unidentified
  REGION 1..15: note = Description of Unknown:signal peptide
MLIIVLLFLATLANS

SEQ ID NO: 61 - protein, 36 aa, unidentified
  REGION 1..36: note = Description of Unknown:signal peptide
MESVSSLFNIFSTIMVNYKSLVLALLSVSNLKYARG

SEQ ID NO: 62 - protein, 18 aa, unidentified
  REGION 1..18: note = Description of Unknown:signal peptide
MFAFYFLTACISLKGVFG

SEQ ID NO: 63 - protein, 23 aa, unidentified
  REGION 1..23: note = Description of Unknown:signal peptide
MRFSTTLATAATALFFTASQVSA

SEQ ID NO: 64 - protein, 16 aa, unidentified
  REGION 1..16: note = Description of Unknown:signal peptide
MKFAYSLLLPLAGVSA

SEQ ID NO: 65 - protein, 16 aa, unidentified
  REGION 1..16: note = Description of Unknown:signal peptide
MKFFAIAALFAAAAVA

SEQ ID NO: 66 - protein, 15 aa, unidentified
  REGION 1..15: note = Description of Unknown:signal peptide
MQFFAVALFATSALA

SEQ ID NO: 67 - protein, 18 aa, unidentified
  REGION 1..18: note = Description of Unknown:signal peptide
MKWVTFISLLFLFSSAYS

SEQ ID NO: 68 - protein, 18 aa, unidentified
  REGION 1..18: note = Description of Unknown:signal peptide
MRSLLILVLCFLPLAALG

SEQ ID NO: 69 - protein, 15 aa, unidentified
  REGION 1..15: note = Description of Unknown:signal peptide
MKVLILACLVALALA

SEQ ID NO: 70 - protein, 18 aa, unidentified
  REGION 1..18: note = Description of Unknown:signal peptide
MFNLKTILISTLASIAVA

SEQ ID NO: 71 - protein, 20 aa, unidentified
  REGION 1..20: note = Description of Unknown:signal peptide
MYRKLAVISAFLATARAQSA

SEQ ID NO: 72 - protein, 42 aa, unidentified
  REGION 1..42: note = Description of Unknown:signal peptide
AYVPSEPWSTLTPDASLESALKDYSQTFGIAIKSLDADKIKR

SEQ ID NO: 73 - protein, 6 aa, unidentified
  REGION 1..6: note = Description of Unknown:signal peptide
ITLPKR

SEQ ID NO: 74 - protein, 15 aa, unidentified
  REGION 1..15: note = Description of Unknown:signal peptide
APLEHQQQHHKHEKR

SEQ ID NO: 75 - protein, 21 aa, unidentified
  REGION 1..21: note = Description of Unknown:signal peptide
APAPKPEDCTTTVQKRHQHKR

SEQ ID NO: 76 - protein, 16 aa, unidentified
  REGION 1..16: note = Description of Unknown:signal peptide
APVAPAEEAANHLHKR

SEQ ID NO: 77 - protein, 28 aa, unidentified
  REGION 1..28: note = Description of Unknown:signal peptide
KNVVDFSRFGDAKISPDDTDLESRERKR

SEQ ID NO: 78 - protein, 18 aa, unidentified
  REGION 1..18: note = Description of Unknown:signal peptide
LPKNVLEEQQAKDDLAKR

SEQ ID NO: 79 - protein, 66 aa, unidentified
  REGION 1..66: note = Description of Unknown:signal peptide
APVNTTTEDETAQIPAEAVIGYLDLEGDFDVAVLPFSNSTNNGLLFINTTIASIAAKEEGVSLDKR

SEQ ID NO: 80 - protein, 4 aa, unidentified
  REGION 1..4: note = Description of Unknown:signal peptide
EAEA

SEQ ID NO: 81 - protein, 13 aa, unidentified
  REGION 1..13: note = Description of Unknown:signal peptide
LEHTHRRGSLVKR

SEQ ID NO: 82 - protein, 33 aa, unidentified
  REGION 1..33: note = Description of Unknown:signal peptide
LDCSGDVFFGYTRGDKTDVHKSQALTAVKNIKR

SEQ ID NO: 83 - protein, 13 aa, unidentified
  REGION 1..13: note = Description of Unknown:signal peptide
MPTSERQQGLEER

SEQ ID NO: 84 - protein, 7 aa, unidentified
  REGION 1..7: note = Description of Unknown:signal peptide
SVINYKR

SEQ ID NO: 85 - protein, 6 aa, unidentified
  REGION 1..6: note = Description of Unknown:signal peptide
QPLEDR

SEQ ID NO: 86 - protein, 6 aa, unidentified
  REGION 1..6: note = Description of Unknown:signal peptide
RGVFRR

(19) United States
(12) Patent Application Publication
(10) Pub. No.: US 2024/0083977 A1
(43) Pub. Date: Mar. 14, 2024

(54) METHODS AND COMPOSITIONS FOR EGG WHITE PROTEIN PRODUCTION

(71) Applicant: Clara Foods Co., Daly City, CA (US)

(72) Inventor: David ANCHEL, San Francisco, CA (US)

(73) Assignee: Clara Foods Co., Daly City, CA (US)

(21) Appl. No.: 18/454,780

(22) Filed: Aug. 23, 2023

Related U.S. Application Data
(63) Continuation of application No. 18/047,953, filed on Oct. 19, 2022.
Application No. 18/047,953 is a continuation of application No. 15/522,986, filed on Apr. 28, 2017, now Pat. No. 11,518,797.
(60) Provisional application No. 62/078,385, filed on Nov. 11, 2014.

(51) Int. Cl. C07K 14/77; A21D 2/26; A23J 1/08; A23J 1/09; A23J 1/18; A23J 3/04; A23J 3/20; A23L 15/00; A23L 33/195; C07K 14/465; C07K 14/79; C12N 15/81; C12P 21/02

(52) U.S. Cl. CPC C07K 14/77; A21D 2/262; A23J 1/08; A23J 1/09; A23J 1/18; A23J 3/04; A23J 3/20; A23L 15/35; A23L 33/195; C07K 14/465; C07K 14/79; C12N 15/815; C12P 21/02; A23V 2002/00

(57) ABSTRACT

Provided herein are compositions, proteins, polynucleotides, expression vectors, host cells, kits, and systems for producing egg white proteins, as well as methods of using the same.

### CROSS-REFERENCE TO RELATED APPLICATIONS

This application is a continuation of U.S. application Ser. No. 18/047,953, filed Oct. 19, 2022, which is a continuation of U.S. application Ser. No. 15/522,986, filed Apr. 28, 2017, now U.S. Pat. No. 11,518,797, which is a National Stage Entry of International Patent Application No. PCT/US15/60147, filed Nov. 11, 2015, which claims the benefit of U.S. Provisional Application No. 62/078,385, filed Nov. 11, 2014, each of which application is incorporated herein by reference in its entirety.

### SEQUENCE LISTING

The instant application contains a Sequence Listing which has been submitted electronically in XML format and is hereby incorporated by reference in its entirety. Said XML copy, created on Sep. 20, 2022, is named 49160701302_SL.xml and is 116,804 bytes in size.

### BACKGROUND OF THE INVENTION

The growing consumer demand for health-conscious fast food options has seen egg white demand at all-time highs in recent years. The success of recently introduced “healthy option” menu items in fast food chains (e.g., McDonald's “egg white delight” breakfast sandwich) and the growing awareness of the link between coronary heart disease and excessive cholesterol consumption have moved consumers to eschew cholesterol-rich egg yolk in favor of relatively high protein, low carbohydrate egg white preparations. This trend has contributed to all-time lows in worldwide dried egg white stocks while the cost of liquid egg whites increased 80% through 2013. This trend is expected to continue, with 2014 July prices double what they were a year earlier and tripling over those in 2012. The dramatic rise in price affects manufacturers of ready-made foodstuffs in particular (e.g., makers of baking mixes), where egg whites are a key ingredient.

Egg-free alternatives to egg protein production aim to offer a solution to the appalling conditions of hen-laying chickens, which ultimately bear the strain of producer's efforts to cut costs and meet a growing worldwide demand. Aside from an increasingly health conscious consumer base, there is widespread recognition of the inhumane conditions of hens subjected to large scale industrial hatchery practices, supported by a scientific consensus of their capability for complex social behaviors and evidence of stress apparent in factory-farmed versus free-roaming egg-laying hens. Such aversion to the inhumane aspects of the industrial hatchery may fuel acceptance and ultimately preference of animal-free egg white alternatives over factory-farmed eggs.

Animal-free egg protein production potentials are not dependent on the productivity of egg-laying hens and are unaffected by market uncertainties due to widespread outbreaks of disease or shortages or price increases in feedstocks. Furthermore, as has been suggested by recent worldwide outbreaks of avian-borne diseases, the risk of avian-to human transmission is exacerbated by farming practices that rely on frequent human contact and the maintenance of dense hen populations. Adoption of an animal-free approach to egg protein production can be viewed as a protective measure against the risk of future avian-to-human disease transmission.

There is a need for alternative egg-free, egg white protein production methods which uncouple production and price from uncertainties in worldwide egg stocks and price variations respectively. Such methods would be attractive options to, for example, fast-food chains which wish to incorporate egg-white options into their menu, as well as manufacturers of egg-white-based food mixes.

### SUMMARY OF THE INVENTION

In one aspect, the present disclosure provides a method of producing an egg white protein composition, the method comprising: recombinantly expressing two or more egg white proteins; and mixing the two or more egg white proteins. In some embodiments, the egg white proteins may be selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. In any one of the preceding embodiments, the recombinantly expressing the two or more egg white proteins may occur in one or more host cells. In any one of the preceding embodiments, the method may further comprise secreting the two or more egg white proteins from the one or more host cells. In any one of the preceding embodiments, the recombinantly expressing the two or more egg white proteins may occur using cell-free protein synthesis. In any one of the preceding embodiments, the method may further comprise adding a food additive to the egg white protein composition. In any one of the preceding embodiments, the method may further comprise desugaring, stabilizing, or removing glucose from the egg white protein composition. In any one of the preceding embodiments, the method may further comprise pasteurizing or ultrapasteurizing the egg white protein composition. In any one of the preceding embodiments, the method may further comprise drying the egg white protein composition. In any one of the preceding embodiments, the method may further comprise enzymatically, chemically, or mechanically digesting one or more of the two or more egg white proteins.

In one aspect, the present disclosure provides a processed consumable product comprising one or more recombinant egg white proteins or fragments thereof. In some embodiments, the one or more egg white proteins may be selected from the group consisting of ovalbumin, ovotransferrin, ovomucoid, G162M F167A ovomucoid, ovoglobulin G2, ovoglobulinG3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. In any one of the preceding embodiments, the processed consumable product may comprise two or more, three or more, four or more, five or more, or six or more egg white proteins or fragments thereof. In any one of the preceding embodiments, the processed consumable product may lack one or more, two or more, three or more, five or more, ten or more, or twenty or more egg white proteins. In any one of the preceding embodiments, the processed consumable product may lack ovomucoid. In any one of the preceding embodiments, the processed consumable product may lack one or more, two or more, three or more, five or more, ten or more, or twenty or more egg yolk proteins. In any one of the preceding embodiments, the processed consumable product may be selected from the group consisting of food product, beverage product, dietary supplement, food additive, pharmaceutical product, hygiene product, and any combination thereof, such as from the group consisting of food product and beverage product.

In one aspect, the present disclosure provides a method of producing a consumable product, the method comprising: recombinantly expressing one or more egg white proteins; and mixing the one or more egg white proteins with one or more ingredients to produce a consumable product. In some embodiments, the one or more ingredients may comprise food additives. In any one of the preceding embodiments, the one or more ingredients may comprise egg white proteins. In any one of the preceding embodiments, the one or more ingredients may comprise recombinant egg white proteins. In any one of the preceding embodiments, the one or more ingredients may not comprise egg white proteins. In any one of the preceding embodiments, the one or more egg white proteins may comprise two or more, three or more, four or more, or five or more egg white proteins.

In one aspect, the present disclosure provides a method for producing an egg white protein or fragment thereof, the method comprising: recombinantly expressing the egg white protein or fragment thereof in a host cell, wherein the host cell may comprise a polynucleotide encoding the egg white protein or fragment thereof, and wherein the egg white protein may be selected from the group consisting of ovoglobulin G2, ovoglobulin G3, α-ovomucin, β-ovomucin, ovoglycoprotein, flavoprotein, ovomacroglobulin, cystatin, and any combination thereof, such as from the group consisting of ovoglobulin G2, ovoglobulin G3, ovoglycoprotein, flavoprotein, ovomacroglobulin, cystatin, and any combination thereof. In some embodiments, the method may further comprise secreting the egg white protein or fragment thereof from the host cell. In any one of the preceding embodiments, the method may further comprise purifying the egg white protein or fragment thereof. In any one of the preceding embodiments, the method may further comprise recombinantly expressing a second egg white protein or fragment thereof in the host cell. In any one of the preceding embodiments, the fragment may comprise at least 10%, 20%, 30%, 40%, or 50% of the egg white protein.

In one aspect, the present disclosure provides a method for producing two or more egg white proteins or fragments thereof, the method comprising recombinantly expressing the two or more egg white proteins or fragments thereof in a host cell. In some embodiments, the host cell may comprise one or more polynucleotides encoding the two or more egg white proteins or fragments thereof. In any one of the preceding embodiments, the method may further comprise secreting the two or more egg white proteins or fragments thereof from the host cell. In any one of the preceding embodiments, the method may further comprise purifying the two or more egg white proteins or fragments thereof. In any one of the preceding embodiments, the two or more egg white proteins may be selected from the group consisting of ovalbumin, ovotransferrin, ovomucoid, G162M F167A ovomucoid, ovoglobulin G2, ovoglobulin G3, α-ovomucin, 3-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof.

In one aspect, the present disclosure provides an isolated recombinant egg white protein selected from the group consisting of ovoglobulin G2, ovoglobulin G3, α-ovomucin, β-ovomucin, ovoglycoprotein, flavoprotein, ovomacroglobulin, cystatin, and any combination thereof, such as from the group consisting of ovoglobulin G2, ovoglobulin G3, ovoglycoprotein, flavoprotein, ovomacroglobulin, cystatin, and any combination thereof. In some embodiments, the isolated recombinant egg white protein may have a glycosylation, acetylation, or phosphorylation pattern different from the egg white protein in an egg white. In any one of the preceding embodiments, the isolated recombinant egg white protein may have a melting temperature different from the egg white protein in an egg white, such as a higher or lower melting temperature relative to the egg white protein in an egg white. In any one of the preceding embodiments, the isolated recombinant egg white protein may comprise one or more amino acid insertions, deletions, or substitutions relative to the egg white protein in an egg white. In any one of the preceding embodiments, the isolated recombinant egg white protein may be selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and any combination thereof.

In one aspect, the present disclosure provides an isolated mutant ovomucoid, comprising tryptophan. In some embodiments, the isolated mutant ovomucoid may be recombinantly expressed. In any one of the preceding embodiments, the isolated mutant ovomucoid may be a complete protein. In any one of the preceding embodiments, the isolated mutant ovomucoid may comprise one or more, two or more, three or more, four or more, five or more, or six or more amino acid insertions or substitutions relative to SEQ ID: NO: 3 when optimally aligned. In any one of the preceding embodiments, the isolated mutant ovomucoid may comprise one or more amino acid substitutions, wherein the amino acid substitutions may comprise one or more, two or more, three or more, four or more, five or more, or six or more tyrosine to tryptophan substitutions. In any one of the preceding embodiments, the isolated mutant ovomucoid may comprise up to four, five, six, or ten amino acid insertions or substitutions relative to SEQ ID: NO: 3 when optimally aligned. In any one of the preceding embodiments, the isolated mutant ovomucoid may comprise one or more, two or more, three or more, or four or more tryptophan residues. In any one of the preceding embodiments, the isolated mutant ovomucoid may comprise one or more, two or more, three or more, or four or more tryptophan residues at the N-terminus or C-terminus. In any one of the preceding embodiments, the isolated mutant ovomucoid may comprise a methionine at position 162 and an alanine at position 167 relative to SEQ ID NO: 3 when optimally aligned. In any one of the preceding embodiments, the isolated mutant ovomucoid may have reduced allergenicity relative to wild-type ovomucoid. In any one of the preceding embodiments, the isolated mutant ovomucoid may have enhanced digestibility relative to wild-type ovomucoid.

In one aspect, the present disclosure provides an egg white protein composition comprising: an isolated recombinant egg white protein or an isolated mutant ovomucoid described herein; and one or more egg white proteins. In some embodiments, the one or more egg white proteins may be recombinantly expressed.

In one aspect, the present disclosure provides an egg white protein composition comprising two or more recombinant egg white proteins.

In some embodiments, for an egg white protein composition described herein, the two or more recombinant egg white proteins may be selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. In any one of the preceding embodiments, the egg white protein composition may comprise ovalbumin. In any one of the preceding embodiments, the egg white protein composition may comprise an isolated recombinant egg white protein described herein. In any one of the preceding embodiments, the egg white protein composition may comprise an isolated mutant ovomucoid described herein. In any one of the preceding embodiments, the egg white proteins may have sequences derived from a single species. In any one of the preceding embodiments, the species may be Gallus gallus domesticus. In any one of the preceding embodiments, the species may be other than Gallus gallus domesticus. In any one of the preceding embodiments, the egg white proteins may have sequences derived from more than one species. In any one of the preceding embodiments, the egg white proteins may have sequences derived from a bird selected from the group consisting of poultry, fowl, waterfowl, game bird, chicken, quail, turkey, duck, ostrich, goose, gull, guineafowl, pheasant, emu, and any combination thereof. In any one of the preceding embodiments, the egg white protein composition may comprise three or more, four or more, or five or more egg white proteins. In any one of the preceding embodiments, the egg white protein composition may comprise up to 5, 10, 15, or 20 egg white proteins. In any one of the preceding embodiments, the egg white protein composition may further comprise water. In any one of the preceding embodiments, the egg white protein composition may have a percentage of water up to 95%. In any one of the preceding embodiments, the egg white protein composition may have a percentage of water within the range from 80% to 95%. In any one of the preceding embodiments, the egg white protein composition may comprise at least 90% protein by dry weight. In any one of the preceding embodiments, the egg white protein composition may further comprise a food additive. In any one of the preceding embodiments, the food additive may be selected from the group consisting of a sweetener, salt, carbohydrate, and any combination thereof.

In any one of the preceding embodiments, the egg white protein composition may lack cholesterol. In any one of the preceding embodiments, the egg white protein composition may comprise less than 5% fat by dry weight. In any one of the preceding embodiments, the egg white protein composition may lack fat, saturated fat, or trans fat. In any one of the preceding embodiments, the egg white protein composition may lack glucose. In any one of the preceding embodiments, the egg white protein composition may lack one or more egg white proteins, such as ovomucoid or flavoprotein. In any one of the preceding embodiments, the one or more egg white proteins may be selected from the group consisting of tenp, clusterin, CH21, VMO-1, vitellogenin, zona pellucida C protein, ovotransferrin BC type, ovoinhibitor precursor, ovomucoid precursor, clusterin precursor, Hep21 protein precursor, ovoglycoprotein precursor, extracellular fatty acid-binding protein, extracellular fatty acid-binding protein precursor, prstaglandin D2 synt may have brain precursor, marker protein, vitellogenin-1, vitellogenin-2, vitellogenin-2 precursor, vitellogenin-3, riboflavin binding protein, hemopexin, serum albumin precursor, apolipoprotein D, ovosecretoglobulin, Hep21, glutathione peroxidase 3, lipocalin-type prostaglandin D synt may have/chondrogenesis-associated lipocalin, apovitellenin-1, dickkopf-related protein 3, gallinacin-11 (VMO-II, β-defensin-11), serum albumin (α-livetin), gallin, secretory trypsin inhibitor, lymphocyte antigen 86, actin, Ig p chain C region, sulfhydryl oxidase 1, histone H4, angiopoietin-like protein 3, ubiquitin, ovocalyxin-32, polymeric immunoglobulin receptor, peptidyl-prolyl-cis/trans isomerase B, aminopeptidase Ey, pleiotrophin, midkine, renin/prorenin receptor, TIMP-2, TIMP-3, histone H2B variants, Ig λ chain, FAMC3 protein, α-enolase, 60S acidic ribosomal protein P1, cytotactin/tenascin, CEPU-1, selenoprotein, elongation factor 1-α 1, epididymal secretory protein, El, 14-3-3 Protein ξ (zeta), olfactomedin-like protein 3, glutathione S-transferase 2, β-2-microglobulin, RGD-CAP, apolipoprotein B, golgi apparatus protein 1, cochlin, proteasome subunit α type-7, apolipoprotein A-I, eukaryotic initiation factor 4A-II, ASPIC/cartilage acidic protein 1, triosephosphate isomerase, proteasome subunit α-type, Ig λ chain C-region, procollagen-lysine 2-oxoglutarate 5-dioxgenase 1, ADP-ribosylation factor 5, calmodulin, protein disulfide-isomerase, annexin I, elongation factor 2, peroxiredoxin-1, HSP70, protein disulfide isomerase A3, calreticulin, 40S ribosomal protein SA/laminin receptor 1, α-Actinin-4, tumor necrosis factor-related apoptosis-inducing ligand, vitamin D-binding protein, semaphorin-3C, endoplasmin, catalase, hepatic α-amylase, transitional ER ATPase, cadherin-1, angiotensin-converting enzyme, bone morphogenetic protein 1, guanine nucleotide-binding protein subunit β2-like 1, histidine ammonia lyase, annexin A2, β-catenin, RAB-GDP dissociation inhibitor, lamin-A, ovocleidin-116, aminopeptidase, HSP90-α, hypoxia up-regulated protein 1, heat shock cognate protein HSP90β, ATP-citrate synt may have, myosin-9, and any combination thereof. In any one of the preceding embodiments, the egg white protein composition may lack two or more, three or more, five or more, ten or more, or twenty or more egg white proteins. In any one of the preceding embodiments, the egg white protein composition is not an egg, egg white, or egg yolk.

In any one of the preceding embodiments, the egg white protein composition may further comprise one or more egg white proteins selected from the group consisting of tenp, clusterin, CH21, VMO-1, vitellogenin, zona pellucida C protein, ovotransferrin BC type, ovoinhibitor precursor, ovomucoid precursor, clusterin precursor, Hep21 protein precursor, ovoglycoprotein precursor, extracellular fatty acid-binding protein, extracellular fatty acid-binding protein precursor, prstaglandin D2 synt may have brain precursor, marker protein, vitellogenin-1, vitellogenin-2, vitellogenin-2 precursor, vitellogenin-3, riboflavin binding protein, hemopexin, serum albumin precursor, apolipoprotein D, ovosecretoglobulin, Hep21, glutathione peroxidase 3, lipocalin-type prostaglandin D synt may have/chondrogenesis-associated lipocalin, apovitellenin-1, dickkopf-related protein 3, gallinacin-11 (VMO-II, β-defensin-11), serum albumin (α-livetin), gallin, secretory trypsin inhibitor, lymphocyte antigen 86, actin, Ig μ chain C region, sulfhydryl oxidase 1, histone H4, angiopoietin-like protein 3, ubiquitin, ovocalyxin-32, polymeric immunoglobulin receptor, peptidyl-prolyl-cis/trans isomerase B, aminopeptidase Ey, pleiotrophin, midkine, renin/prorenin receptor, TIMP-2, TIMP-3, histone H2B variants, Ig λ chain, FAMC3 protein, α-enolase, 60S acidic ribosomal protein P1, cytotactin/tenascin, CEPU-1, selenoprotein, elongation factor 1-α 1, epididymal secretory protein, El, 14-3-3 Protein ξ (zeta), olfactomedin-like protein 3, glutathione S-transferase 2, β-2-microglobulin, RGD-CAP, apolipoprotein B, golgi apparatus protein 1, cochlin, proteasome subunit α type-7, apolipoprotein A-I, eukaryotic initiation factor 4A-II, ASPIC/cartilage acidic protein 1, triosephosphate isomerase, proteasome subunit α-type, Ig λ chain C-region, procollagen-lysine 2-oxoglutarate 5-dioxgenase 1, ADP-ribosylation factor 5, calmodulin, protein disulfide-isomerase, annexin I, elongation factor 2, peroxiredoxin-1, HSP70, protein disulfide isomerase A3, calreticulin, 40S ribosomal protein SA/laminin receptor 1, α-Actinin-4, tumor necrosis factor-related apoptosis-inducing ligand, vitamin D-binding protein, semaphorin-3C, endoplasmin, catalase, hepatic α-amylase, transitional ER ATPase, cadherin-1, angiotensin-converting enzyme, bone morphogenetic protein 1, guanine nucleotide-binding protein subunit β2-like 1, histidine ammonia lyase, annexin A2, β-catenin, RAB-GDP dissociation inhibitor, lamin-A, ovocleidin-116, aminopeptidase, HSP90-α, hypoxia up-regulated protein 1, heat shock cognate protein HSP90 β, ATP-citrate synt may have, and myosin-9, and any combination thereof.

In any one of the preceding embodiments, the egg white protein composition may have a pH within the range from 6 to 10. In any one of the preceding embodiments, the egg white protein composition may have a foam height within the range from 10 mm to 60 mm, such as from 30 mm to 60 mm. In any one of the preceding embodiments, the egg white protein composition may have a foam height of at least 30 mm. In any one of the preceding embodiments, the egg white protein composition may have a foam height greater than a foam height of an egg white. In any one of the preceding embodiments, the egg white protein composition may have a foam seep up to 10 mm or up to 5 mm at 30 minutes after whipping. In any one of the preceding embodiments, the egg white protein composition may have a foam seep less than a foam seep of an egg white at 30 minutes after whipping. In any one of the preceding embodiments, the egg white protein composition may have a foam strength within the range from 30 g to 100 g, such as from 40 g to 100 g. In any one of the preceding embodiments, the egg white protein composition may have a foam strength greater than a foam strength of an egg white. In any one of the preceding embodiments, the egg white protein composition may have a gel strength within the range from 100 g to 1500 g, from 500 g to 1500 g, or from 700 g to 1500 g. In any one of the preceding embodiments, the egg white protein composition may have a gel strength greater than a gel strength of an egg white. In any one of the preceding embodiments, the egg white protein composition may have a shelf life of at least one, two, three, or six months. In any one of the preceding embodiments, the egg white protein composition may have reduced allergenicity relative to an egg white. In any one of the preceding embodiments, the egg white protein composition may be a liquid. In any one of the preceding embodiments, the egg white protein composition may be a solid or powder. In any one of the preceding embodiments, the egg white protein composition may be frozen.

In one aspect, the present disclosure provides a polynucleotide encoding an isolated recombinant egg white protein or isolated mutant ovomucoid described herein. In some embodiments, the polynucleotide may be codon optimized. In any one of the preceding embodiments, the polynucleotide may be DNA or RNA. In any one of the preceding embodiments, the polynucleotide may further encode a signal peptide. In any one of the preceding embodiments, the signal peptide may be at the N-terminus of the egg white protein or polypeptide. In any one of the preceding embodiments, the signal peptide may be selected from the group consisting of acid phosphatase, albumin, alkaline extracellular protease, α-mating factor, amylase, β-casein, carbohydrate binding module family 21-starch binding domain, carboxypeptidase Y, cellobiohydrolase I, dipeptidyl protease, glucoamylase, heat shock protein, hydrophobin, inulase, invertase, killer protein or killer toxin, leucine-rich artificial signal peptide CLY-L8, lysozyine, phytohemagglutinin, maltose binding protein, P-factor, Pichia pastoris Dse, Pichia pastoris Exg, Pichia pastoris Pirl, Pichia pastoris Scw, Pir4, and any combination thereof. In any one of the preceding embodiments, the signal peptide may be selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and any combination thereof. In any one of the preceding embodiments, the signal peptide may be up to 100 amino acids in length. In any one of the preceding embodiments, the polynucleotide may further encode a signal peptidase cleavage or recognition site. In any one of the preceding embodiments, the signal peptidase may be selected from the group consisting of KEX2, Krpl, Enterokinase (EKT), thrombin, factor Xa (FXa), Tobacco Etch Virus (TEV), 3C Prescission, and any combination thereof.

In one aspect, the present disclosure provides an expression vector comprising a polynucleotide described herein. In some embodiments, the expression vector may further comprise a promoter. In any one of the preceding embodiments, the promoter may be a constitutive promoter, an inducible promoter, or a hybrid promoter. In any one of the preceding embodiments, the promoter may be selected from the group consisting of acu-5, adhl+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbhl), ccg-1, cDNA1, cellular filament polypeptide (cfp), cpc-2, ctr4+, CUP1, dihydroxyacetone synt may have (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, me1O, MET3, methanol oxidase (MOX), nmtl, NSP, pcbC, PET9, peroxin 8 (PEX8), phosphoglycerate kinase (PGK, PGK1), phol, PHO5, PHO89, phosphatidylinositol synt may have (PIS1), PYK1, pyruvate kinase (pkil), RPS7, sorbitol dehydrogenase (SDH), 3-phosphoserine aminotransferase (SERI), SSA4, SV40, TEF, translation elongation factor 1 alpha (TEF1), THI11 homoserine kinase (THR1), tpi, TPS1, triose phosphate isomerase (TPI1), XRP2, YPT1, and any combination thereof. In any one of the preceding embodiments, the expression vector may further comprise an auxotrophic marker. In any one of the preceding embodiments, the auxotrophic marker may be selected from the group consisting of ade1, arg4, his4, ura3, met2, and any combination thereof. In any one of the preceding embodiments, the expression vector may further comprise a selectable marker. In any one of the preceding embodiments, the selectable marker may be a resistance gene. In any one of the preceding embodiments, the resistance gene may confer resistance to zeocin, ampicillin, blasticidin, kanamycin, nurseothricin, chloroamphenicol, tetracycline, triclosan, ganciclovir, or any combination thereof. In any one of the preceding embodiments, the expression vector may comprise a plasmid.

In one aspect, the present disclosure provides a host cell transformed to express one or more heterologous egg white proteins, wherein the host cell are not selected from the group consisting of Escherichia coli, Pichia pastoris, rice, Aspergillus niger, Aspergillus oryzae, Acremonium chrysogenum, Saccharomyces cerevisiae, insect, mice, corn, Pseudozyma, tobacco, zebrafish, and any combination thereof.

In one aspect, the present disclosure provides a host cell transformed to express one or more heterologous egg white proteins, wherein the one or more egg white proteins are not selected from the group consisting of ovalbumin, ovotransferrin, lysozyme, ovostatin, ovomucoid, ovoinhibitor, avidin, and any combination thereof.

In one aspect, the present disclosure provides a host cell comprising a polynucleotide described herein.

In one aspect, the present disclosure provides a host cell comprising an expression vector described herein. In some embodiments, the expression vector may be genomically integrated. In any one of the preceding embodiments, the host cell may comprise multiple copies of the expression vector.

In any one of the preceding embodiments, the host cell may be selected from the group consisting of bacteria, fungi, plant, insect, mammalian, and any combination thereof. In any one of the preceding embodiments, the fungi may be a yeast or filamentous fungi. In any one of the preceding embodiments, the yeast may be selected from the group consisting of Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, and any combination thereof. In any one of the preceding embodiments, the fungi may be selected from the group consisting of Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, Trichoderma vireus, and any combination thereof. In any one of the preceding embodiments, the host cell may be selected from the group consisting of Aspergillus oryzae, Bacillus subtilis, Escherichia coli, Myceliophthora thermophila, Neurospora crassa, Pichia pastoris, and any combination thereof. In any one of the preceding embodiments, the host cell may be approved as generally regarded as safe by the U.S. Food and Drug Administration. In any one of the preceding embodiments, the host cell may be auxotrophic.

In one aspect, the present disclosure provides a cell culture comprising a host cell described herein.

In one aspect, the present disclosure provides a method for making a consumable product, the method comprising substituting a portion of an egg-based ingredient with an isolated recombinant egg white protein, isolated mutant ovomucoid, or egg white protein composition described herein.

In one aspect, the present disclosure provides a method for making a consumable product, the method comprising adding an isolated recombinant egg white protein, isolated mutant ovomucoid, or egg white protein composition described herein.

In one aspect, the present disclosure provides a method of using a recombinant egg white protein as a processing agent to make a processed consumable product. In some embodiments, the method may further comprise removing the recombinant egg white protein.

In one aspect, the present disclosure provides a method of using an isolated recombinant egg white protein, isolated mutant ovomucoid, or egg white protein composition described herein as a processing agent to make a processed consumable product. In some embodiments, the method may further comprise removing the isolated recombinant egg white protein, isolated mutant ovomucoid, or egg white protein composition.

In any one of the preceding embodiments, the processing agent acts as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a consumable product comprising an isolated recombinant egg white protein, isolated mutant ovomucoid, or egg white protein composition described herein. In some embodiments, the consumable product may be selected from the group consisting of food product, beverage product, pharmaceutical product, hygiene product, and any combination thereof.

In one aspect, the present disclosure provides a method of using a recombinant egg white protein as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a method of using an isolated recombinant egg white protein, isolated mutant ovomucoid, or egg white protein composition described herein as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a method for diagnosing a food allergy, the method comprising introducing an isolated recombinant egg white protein, isolated mutant ovomucoid, or egg white protein composition described herein to a subject. In some embodiments, the introducing may be performed using a skin prick test, blood test, or oral food challenge.

In one aspect, the present disclosure provides a method for treating a food allergy, the method comprising substituting an egg white allergen with an isolated recombinant egg white protein, isolated mutant ovomucoid, or egg white protein composition described herein or increasing a tolerance to an egg white allergen of a subject by consuming an isolated recombinant egg white protein, isolated mutant ovomucoid, or egg white protein composition described herein.

In one aspect, the present disclosure provides a method for inhibiting malolactic fermentation in wine, the method comprising providing an egg white lysozyme to wine. In some embodiments, the egg white lysozyme may be recombinantly expressed.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an amino acid sequence of ovalbumin (SEQ ID NO: 1).

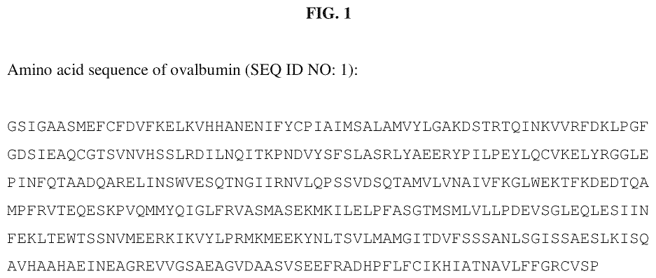

FIG. 2 is an amino acid sequence of ovotransferrin (SEQ ID NO: 2).

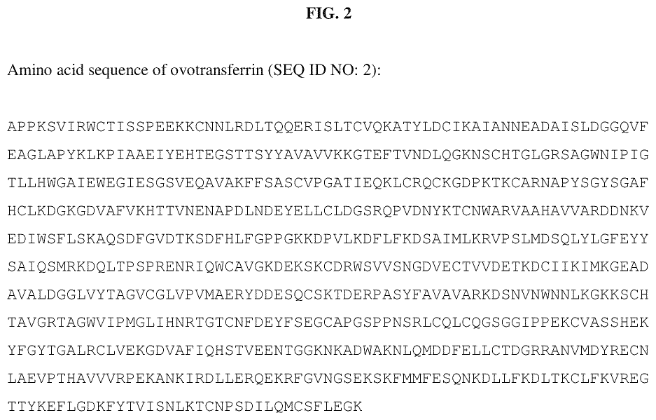

FIG. 3 is an amino acid sequence of ovomucoid (SEQ ID NO: 3).

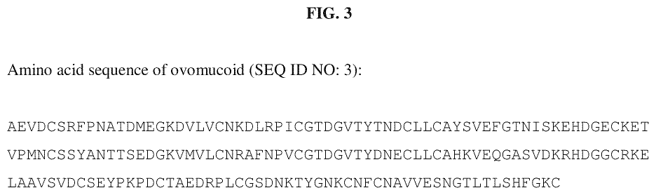

FIG. 4 is an amino acid sequence of G162M F167A ovomucoid (SEQ ID NO: 4).

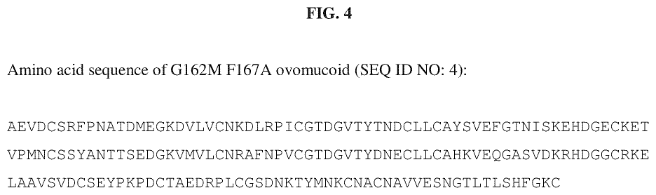

FIG. 5 is an amino acid sequence of ovoglobulin G2 (SEQ ID NO: 5).

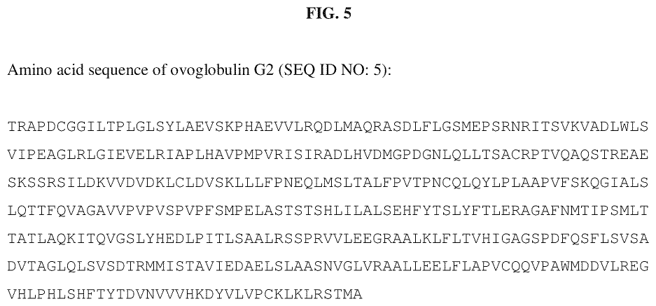

FIG. 6 is an amino acid sequence of ovoglobulin G3 (SEQ ID NO: 6).

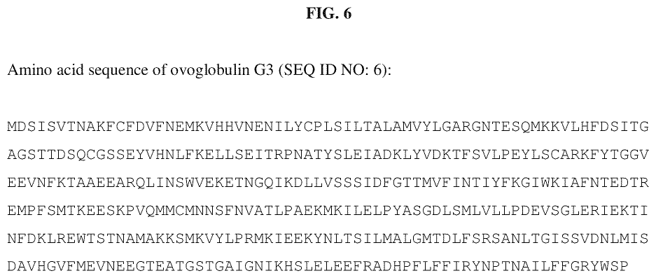

FIG. 7 is an amino acid sequence of α-ovomucin (SEQ ID NO: 7).

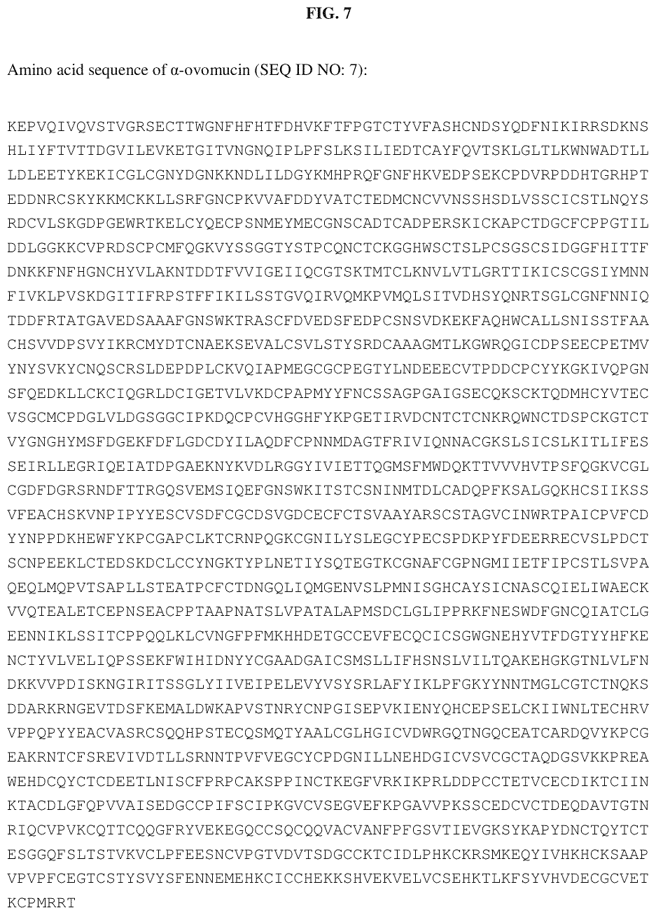

FIG. 8 is a partial amino acid sequence of β-ovomucin (SEQ ID NO: 8).

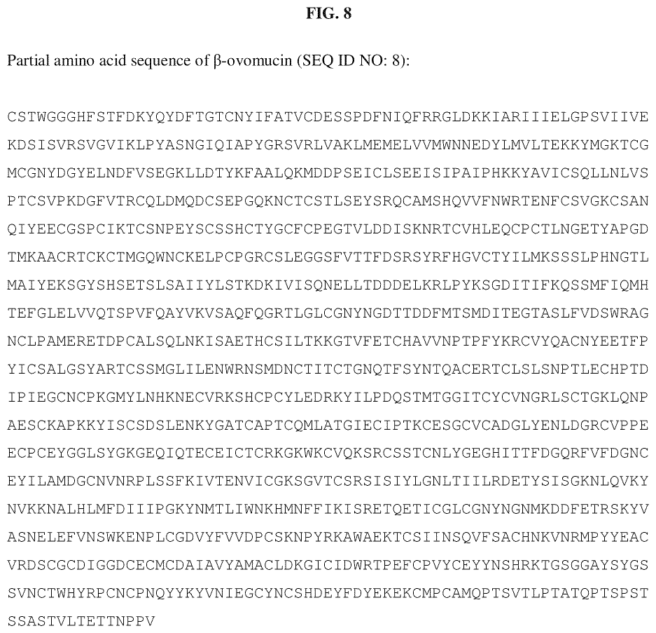

FIG. 9 is an amino acid sequence of lysozyme (SEQ ID NO: 9).

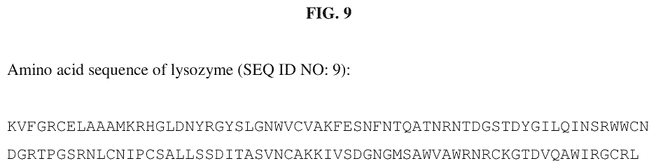

FIG. 10 is an amino acid sequence of ovoinhibitor (SEQ ID NO: 10).

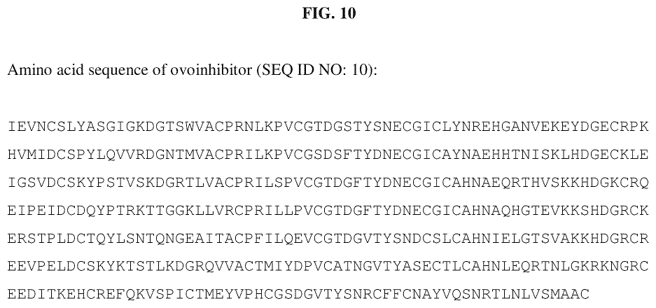

FIG. 11 is an amino acid sequence of cystatin (SEQ ID NO: 11).

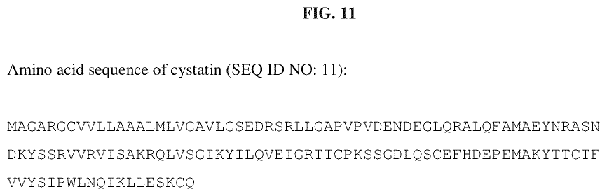

FIG. 12 is an amino acid sequence of ovalbumin related protein X (SEQ ID NO: 12).

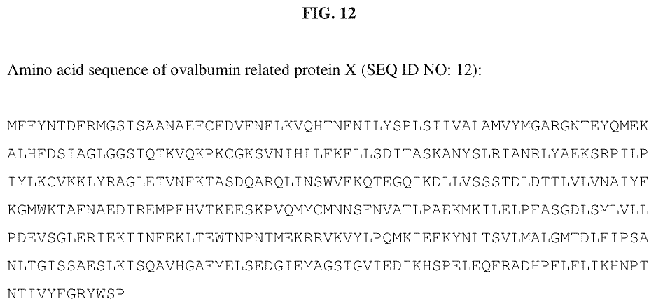

FIG. 13 is an amino acid sequence of ovalbumin related protein Y (SEQ ID NO: 13).

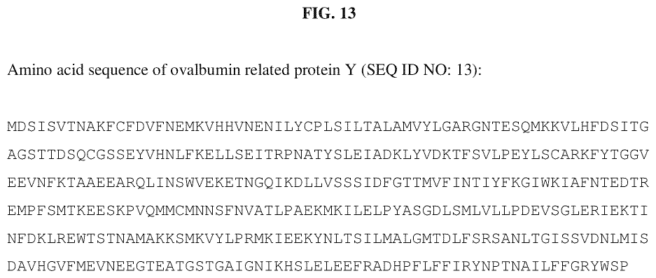

FIG. 14 shows a schematic diagram of an ovoglobulin expression vector, in accordance with examples.

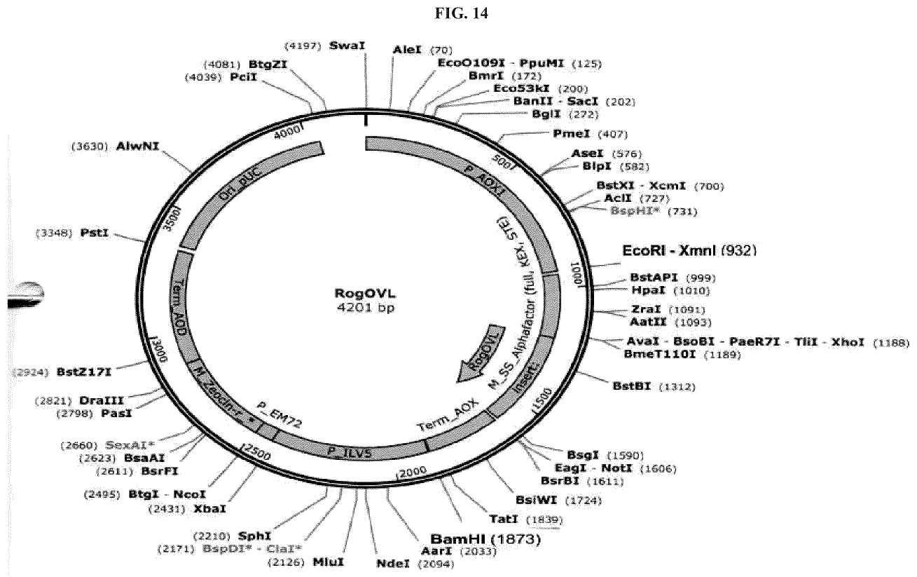

FIG. 15 shows a schematic diagram of a lysozyme expression vector, in accordance with examples.

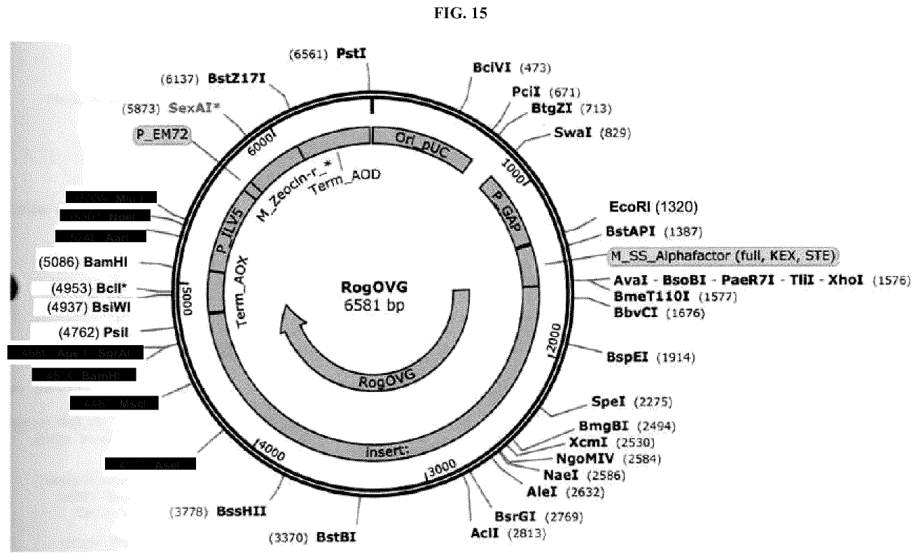

FIG. 16 shows a gel image of recombinant ovalbumin with a protein ladder, in accordance with examples.

FIG. 17 shows a gel image of recombinant ovomucoid with a protein ladder, in accordance with examples.

FIG. 18 shows a schematic diagram of foam strength of an egg white protein composition and an egg white, in accordance with examples.

### DETAILED DESCRIPTION OF THE INVENTION

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polynucleotide”, “nucleotide”, “nucleotide sequence”, “nucleic acid”, and “oligonucleotide” are used interchangeably. They refer to a polymeric form of nucleotides of any length, including deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, expression vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a detectable label.

“Expression” refers to the process by which a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide”, and “protein” are used interchangeably herein to refer to polymers of amino acids of any length. The polymer may be linear or branched, may comprise modified amino acids, and may be interrupted by non amino acids. The terms also encompass an amino acid polymer that has been modified, for example, by disulfide bond formation, glycosylation, lipidation, acetylation, phosphorylation, or any other manipulation, such as conjugation with a detectable label.

As used herein the term “amino acid” includes natural and/or unnatural or synthetic amino acids, including glycine, cysteine, and both the D or L optical isomers, and amino acid analogs and peptidomimetics. In some embodiments, an amino acid is a proteinogenic, natural, standard, non-standard, non-canonical, essential, non-essential, or non-natural amino acid. In some embodiments, an amino acid has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, an amino acid has a nucleophilic or electrophilic side chain.

“Control” refers to an alternative subject or sample used in an experiment for comparison purpose. In some embodiments, a control comprises egg white from a chicken egg.

The terms “determining”, “measuring”, “evaluating”, “assessing”, “assaying”, and “analyzing” can be used interchangeably herein to refer to any form of measurement, and include determining if an element is present or not (for example, detection). These terms can include both quantitative and/or qualitative determinations. Assessing may be relative or absolute. “Detecting the presence of can include determining the amount of something present and/or determining whether it is present or absent.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 96%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Sequence identity, such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm (see e.g., the BLAST alignment tool available at blast.ncbi.nlm.nih.gov/Blast.cgi, optionally with default settings), and the Smith-Waterman algorithm (see e.g., the EMBOSS Water aligner available at www.ebi.ac.uk/Tools/psa/emboss_water/nucleotide.htrnl, optionally with default settings). Optimal alignment may be assessed using any suitable parameters of a chosen algorithm, including default parameters.

In general, “sequence identity” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (e.g., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%. In general, an exact match indicates 100% identity over the length of the reference sequence.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

The term “mammal” includes humans and both domestic animals such as laboratory animals and household pets (e.g., cats, dogs, swine, cattle, sheep, goats, horses, rabbits), and non-domestic animals such as wildlife and the like. Mammals include, but are not limited to, murines, simians, humans, farm animals, sport animals, domesticated animals, and pets. Tissues, cells, and their progeny of a biological entity obtained in vivo or cultured in vitro are also encompassed.

The term “bird” includes both domesticated birds and non-domesticated birds such as wildlife and the like. Birds include, but are not limited to, poultry, fowl, waterfowl, game bird, ratite (e.g., flightless bird), chicken (Gallus gallus domesticus), quail, turkey, duck, ostrich (Struthio camelus), Somali ostrich (Struthio molybdophanes), goose, gull, guineafowl, pheasant, emu (Dromaius novaehollandiae), American rhea (Rhea americana), Darwin's rhea (Rhea pennata), and kiwi. Tissues, cells, and their progeny of a biological entity obtained in vivo or cultured in vitro are also encompassed. A bird may lay eggs.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

In certain embodiments, the proteins or compounds disclosed herein are isotopically labeled. Isotopically-labeled proteins or compounds (e.g., an isotopologue) may have one or more atoms replaced by an atom having a different atomic mass or mass number. Non-limiting examples of isotopes that can be incorporated into the disclosed compounds include isotopes of hydrogen, carbon, nitrogen, oxygen, phosphorous, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³-I, and ¹²⁵I, respectively. Certain isotopically-labeled compounds, for example, those incorporating a stable isotope, are useful in mass spectrometry studies. For instance, a stable isotopic protein may be used as a reference standard in a mass spectrometry based assay. Certain isotopically-labeled compounds, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. These radiolabeled compounds could be useful to help determine or measure the effectiveness of the compounds, by characterizing, for example, the site or mode of action, or binding affinity to a pharmacologically important site of action. Substitution with heavier isotopes such as deuterium (²H) may afford certain therapeutic advantages resulting from greater metabolic stability, for example, increased in vivo half-life or reduced dosage requirements, and hence are preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described herein using an appropriate isotopically-labeled reagent in place of the non-labeled reagent.

“Optional” and “optionally” mean that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

As used herein, the term “consumable product” refers to a product, which comprises an isolated recombinant egg white protein or egg white protein composition and other ingredients and may be consumed (e.g., by eating, chewing, drinking, tasting, ingesting, or swallowing). Consumable products include food products, beverage products, dietary supplements, food additives, pharmaceutical products, and hygiene products, as non-limiting examples. Food products include, but are not limited to, baked goods (e.g., cake, muffin, cookie, bread, bagel, pastry, doughnut), scramble, omelette, quiche, pasta, noodle, crepe, waffle, dough, batter, cookie dough, meatloaf, meatball, hamburger, animal feed, fruits, vegetables, tofu, bean curd, cheese, seafood, meat, ice cream, mayonnaise, custard, pudding, souffle, emulsion, foam, meringue, frosting, confectionery, marshmallow, marzipan, soup, condiments, sauces, spices, dairy products, and dressings. Beverage products include, but are not limited to, soft drink, flavored water, juice, sports drink, energy drink, smoothie, shake, alcoholic beverage (e.g., wine, sake, beer, spirits), cocktail, liqueur, carbonated beverage, caffeinated beverage, coffee, cocoa, tea, eggnog, and dairy drinks. Dietary supplements include multivitamins, whole food supplements, diet supplements, herbal supplement, protein blend, mass gainer, ready to drink protein, protein bar, protein shake, protein powder, protein shot, protein isolate, energy bar, energy gel, energy chew, energy formula, endurance formula, energy supplement, nutritional supplement, sports nutritional supplement, infant formula (e.g., powder or liquid), and meal replacement. Pharmaceutical products include, but are not limited to, cough syrups, capsules, and tablets. Hygiene products include, but are not limited to, cosmetics, skin care, beauty products, shampoo, conditioner, lotion, cream, face wash, tooth paste, chewing gum, and mouth wash.

Processing of a consumable product to form a processed consumable product may include, but is not limited to, freezing, chilling, heating, baking, roasting, broiling, boiling, blanching, packaging, canning, bleaching, enriching, drying, pressing, grinding, mixing, parcooking, cooking, proofing, marinating, cutting, slicing, dicing, crushing, shredding, chopping, shaking, coring, spiralizing, rolling, juicing, straining, filtering, kneading, whisking, beating, whipping, grating, stuffing, peeling, deseeding, smoking, curing, salting, preserving, pickling, fermenting, homogenizing, pasteurizing, sterilizing, stabilizing, blending, pureeing, fortifying, refining, hydrogenating, aging, extending shelf life, or adding enzymes.

As used herein, the term “solvent” refers to a liquid, which may be mixed with or used to dissolve a composition or one or more components of a composition such as a protein. Non-limiting examples of a solvent include water, ethanol, and isopropanol. The solvent can be potable. The solvent can be water. Non-limiting examples of water include purified water, distilled water, double distilled water, deionized water, distilled deionized water, drinking water, well water, tap water, spring water, bottled water, carbonated water, mineral water, flavored water, or any combination thereof. A solvent may be a combination of two or more distinct solvents.

Overview of Animal-Free Egg-White Production System

Provided herein are methods to produce protein components of egg white through recombinant expression in a host cell (e.g., yeast) and/or to purify egg white proteins from secretions of in vitro cultured oviduct cells. The purified proteins can be stored using established methods (e.g., spray drying), packaged as a powdered product, or sold in reconstituted form as an egg white protein composition that resembles animal-derived egg whites in consistency, taste, functional properties, and/or appearance. Different formulations of protein constituents of the egg white protein composition can be achieved as the absence or abundance of individual constituents can be adjusted independently. In one embodiment, ovomucoid, a major food allergen in egg white that can cause immediate food-hypersensitivity in children, can be eliminated in the final formulation, or modified genetically and/or glycosylated to produce a reduced allergenicity egg white product.

Bird eggs are a common food source and a versatile ingredient in cooking. Eggs generally contain an eggshell, membrane, egg white, and egg yolk. The egg white or albumen contains approximately 10% proteins, 88% water, and 1-2% carbohydrates, minerals, and lipids. Egg white proteins may include, but are not limited to, ovalbumin (˜54%), ovotransferrin or conalbumin (˜12%), ovomucoid (˜11%), ovoglobulin G2 (˜4%), ovoglobulin G3 (˜4%), ovomucin (˜3.5%), lysozyme (˜3.4%), ovoinhibitor (˜1.5%), ovoglycoprotein (˜1.0%), flavoprotein or ovoflavoprotein (˜0.8%), ovomacroglobulin (˜0.5%), cystatin (˜0.05%), avidin (˜0.05%), ovostatin, ovalbumin related protein X, ovalbumin related protein Y, tenp, clusterin, CH21, VMO-1, vitellogenin, zona pellucida C protein, ovotransferrin BC type, ovoinhibitor precursor, ovomucoid precursor, clusterin precursor, Hep21 protein precursor, ovoglycoprotein precursor, extracellular fatty acid-binding protein, extracellular fatty acid-binding protein precursor, prstaglandin D2 synthase brain precursor, marker protein, vitellogenin-1, vitellogenin-2, vitellogenin-2 precursor, vitellogenin-3, riboflavin binding protein, hemopexin, serum albumin precursor, apolipoprotein D, ovosecretoglobulin, Hep21, glutathione peroxidase 3, lipocalin-type prostaglandin D synthase/chondrogenesis-associated lipocalin, apovitellenin-1, dickkopf-related protein 3, gallinacin-11 (VMO-II, β-defensin-11), serum albumin (α-livetin), gallin, secretory trypsin inhibitor, lymphocyte antigen 86, actin, Ig chain C region, sulfhydryl oxidase 1, histone H4, angiopoietin-like protein 3, ubiquitin, ovocalyxin-32, polymeric immunoglobulin receptor, peptidyl-prolyl-cis/trans isomerase B, aminopeptidase Ey, pleiotrophin, midkine, renin/prorenin receptor, TIMP-2, TIMP-3, histone H2B variants, Ig λ chain, FAMC3 protein, α-enolase, 60S acidic ribosomal protein P1, cytotactinitenascin, CEPU-1, selenoprotein, elongation factor 1-α 1, epididymal secretory protein, El, 14-3-3 Protein ξ (zeta), olfactomedin-like protein 3, glutathione S-transferase 2, β-2-microglobulin, RGD-CAP, apolipoprotein B, golgi apparatus protein 1, cochlin, proteasome subunit α type-7, apolipoprotein A-I, eukaryotic initiation factor 4A-II, ASPIC/cartilage acidic protein 1, triosephosphate isomerase, proteasome subunit α-type, Ig λ chain C-region, procollagen-lysine 2-oxoglutarate 5-dioxgenase 1, ADP-ribosylation factor 5, calmodulin, protein disulfide-isomerase, annexin I, elongation factor 2, peroxiredoxin-1, HSP70, protein disulfide isomerase A3, calreticulin, 40S ribosomal protein SA/laminin receptor 1, α-Actinin-4, tumor necrosis factor-related apoptosis-inducing ligand, vitamin D-binding protein, semaphorin-3C, endoplasmin, catalase, hepatic α-amylase, transitional ER ATPase, cadherin-1, angiotensin-converting enzyme, bone morphogenetic protein 1, guanine nucleotide-binding protein subunit β2-like 1, histidine ammonia lyase, annexin A2, β-catenin, RAB-GDP dissociation inhibitor, lamin-A, ovocleidin-116, aminopeptidase, HSP90-α, hypoxia up-regulated protein 1, heat shock cognate protein HSP90β, ATP-citrate synthase, and myosin-9.

In one aspect, the present disclosure provides a method of producing an egg white protein composition, the method comprising: recombinantly expressing two or more egg white proteins; and mixing the two or more egg white proteins. In some cases, the egg white proteins are selected from the group consisting of ovalbumin, ovotransferrin, ovomucoid, G162M Fl 67A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. The recombinantly expressing the two or more egg white proteins may occur in one or more host cells. The method may further comprise secreting the two or more egg white proteins from the one or more host cells. The recombinantly expressing the two or more egg white proteins may occur using cell-free protein synthesis. The method may further comprise adding a food additive to the egg white protein composition. The method may further comprise desugaring, stabilizing, or removing glucose from the egg white protein composition. The method may further comprise pasteurizing or ultrapasteurizing the egg white protein composition. The method may further comprise drying the egg white protein composition. The method may further comprise enzymatically, chemically, or mechanically digesting one or more of the two or more egg white proteins. Treatment of one or more of the egg white proteins may improve functional properties of the composition, for example, providing enhanced foaming or solubility.

In one aspect, the present disclosure provides a processed consumable product comprising one or more recombinant egg white proteins or fragments thereof. In some cases, the one or more egg white proteins are selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. In some cases, the processed consumable product comprises two or more, three or more, four or more, five or more, or six or more egg white proteins or fragments thereof. In some cases, the processed consumable product lacks one or more, two or more, three or more, five or more, ten or more, twenty or more, or fifty or more egg white proteins. The processed consumable product may lack ovomucoid. In some cases, the processed consumable product may lack one or more, two or more, three or more, five or more, ten or more, or twenty or more egg yolk proteins. In some cases, the processed consumable product is selected from the group consisting of food product, beverage product, dietary supplement, food additive, pharmaceutical product, hygiene product, and any combination thereof, such as from the group consisting of food product, beverage product, and any combination thereof.

In one aspect, the present disclosure provides a method of producing a consumable product, the method comprising: recombinantly expressing one or more egg white proteins; and mixing the one or more egg white proteins with one or more ingredients to produce a consumable product. In some cases, the one or more ingredients comprise food additives, egg white proteins, or recombinant egg white proteins. In some cases, the one or more ingredients do not comprise egg white proteins. In some cases, the one or more egg white proteins may comprise two or more, three or more, four or more, or five or more egg white proteins.

In one aspect, the present disclosure provides a method for producing an egg white protein or fragment thereof, the method comprising: recombinantly expressing the egg white protein or fragment thereof in a host cell, wherein the host cell comprises a polynucleotide encoding the egg white protein or fragment thereof, and wherein the egg white protein is selected from the group consisting of ovoglobulin G2, ovoglobulin G3, α-ovomucin, β-ovomucin, ovoglycoprotein, flavoprotein, ovomacroglobulin, cystatin, and any combination thereof, such as from the group consisting of ovoglobulin G2, ovoglobulin G3, ovoglycoprotein, flavoprotein, ovomacroglobulin, cystatin, and any combination thereof. The method may further comprise secreting the egg white protein or fragment thereof from the host cell. The method may further comprise purifying the egg white protein or fragment thereof. The method may further comprise recombinantly expressing a second egg white protein or fragment thereof in the host cell. In some cases, the fragment comprises at least 10%, 20%, 30%, 40%, or 50% of the egg white protein.

In some cases, a fragment of a protein may be about or at least 10, 50, 100, 200, 300, 400, 500, 600, 700, 800, 900, 1000, 1100, 1200, 1300, 1400, 1500, 1600, 1700, 1800, 1900, or 2000 amino acids in length. In some cases, a fragment of a protein may be up to 10, 50, 100, 200, 300, 400, 500, 600, 700, 800, 900, 1000, 1100, 1200, 1300, 1400, 1500, 1600, 1700, 1800, 1900, or 2000 amino acids in length.

In some cases, a fragment of a protein may be about or at least 1%, 5%, 10%, 15%, 20%, 25%, 30%, 35%, 40%, 45%, 50%, 55%, 60%, 65%, 70%, 75%, 80%, 85%, 90%, 95%, or 100% of the protein. In some cases, a fragment of a protein may be up to 1%, 5%, 10%, 15%, 20%, 25%, 30%, 35%, 40%, 45%, 50%, 55%, 60%, 65%, 70%, 75%, 80%, 85%, 90%, 95%, or 100% of the protein. In some cases, a fragment of a protein may be about or at least 10, 50, 100, 200, 300, 400, 500, 600, 700, 800, 900, or 1000 kDa. In some cases, a fragment of a protein may be up to 10, 50, 100, 200, 300, 400, 500, 600, 700, 800, 900, or 1000 kDa.

In one aspect, the present disclosure provides a method for producing two or more egg white proteins or fragments thereof, the method comprising recombinantly expressing the two or more egg white proteins or fragments thereof in a host cell. The host cell may comprise one or more polynucleotides encoding the two or more egg white proteins or fragments thereof. The method may further comprise secreting the two or more egg white proteins or fragments thereof from the host cell. The method may further comprise purifying the two or more egg white proteins or fragments thereof. In some cases, the two or more egg white proteins are selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof.

In one aspect, the present disclosure provides an isolated recombinant egg white protein selected from the group consisting of ovoglobulin G2, ovoglobulin G3, α-ovomucin, β-ovomucin, ovoglycoprotein, flavoprotein, ovomacroglobulin, cystatin, and any combination thereof, such as from the group consisting of ovoglobulin G2, ovoglobulin G3, ovoglycoprotein, flavoprotein, ovomacroglobulin, cystatin, and any combination thereof. The isolated recombinant egg white protein may have a glycosylation, acetylation, or phosphorylation pattern different from the egg white protein in an egg white. The isolated recombinant egg white protein may have a melting temperature different from the egg white protein in an egg white, such as a higher or lower melting temperature relative to the egg white protein in an egg white. In some cases, the isolated recombinant egg white protein has a melting temperature of about or at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some cases, the isolated recombinant egg white protein has a melting temperature of up to 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some cases, the isolated recombinant egg white protein may comprise one or more amino acid insertions, deletions, or substitutions relative to the egg white protein in an egg white. In some cases, an isolated recombinant egg white protein may have about or at least 0, 1, 2, 3, 4, 5, 6, 7, 8, 9, 10, 15, 20, 25, 30, 40, 50, 60, 70, 80, 90, 100, 150, 200, 250, 300, 350, 400, 450, 500, 600, 700, 800, 900, or 1000 amino acid insertions, deletions, and/or substitutions relative to the egg white protein in an egg white. In some cases, an isolated recombinant egg white protein may have up to 0, 1, 2, 3, 4, 5, 6, 7, 8, 9, 10, 15, 20, 25, 30, 40, 50, 60, 70, 80, 90, 100, 150, 200, 250, 300, 350, 400, 450, 500, 600, 700, 800, 900, or 1000 amino acid insertions, deletions, and/or substitutions relative to the egg white protein in an egg white. In some cases, the isolated recombinant egg white protein is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and any combination thereof.

In one aspect, the present disclosure provides an isolated mutant ovomucoid, comprising tryptophan. The isolated mutant ovomucoid may be recombinantly expressed. The isolated mutant ovomucoid may be a complete protein. In some cases, the isolated mutant ovomucoid comprises one or more, two or more, three or more, four or more, five or more, or six or more amino acid insertions or substitutions relative to SEQ ID: NO: 3 when optimally aligned. In some cases, the isolated mutant ovomucoid comprises one or more amino acid substitutions, wherein the amino acid substitutions comprise one or more, two or more, three or more, four or more, five or more, or six or more tyrosine to tryptophan substitutions (e.g., at position 37, 46, 73, 102, 141, or 161 relative to SEQ ID NO: 3 or SEQ ID NO: 4 when optimally aligned). In some cases, the isolated mutant ovomucoid comprises up to four, five, six, or ten amino acid insertions or substitutions relative to SEQ ID: NO: 3 when optimally aligned. In some cases, the isolated mutant ovomucoid comprises one or more, two or more, three or more, or four or more tryptophan residues. In some cases, the isolated mutant ovomucoid comprises one or more, two or more, three or more, or four or more tryptophan residues at the N-terminus or C-terminus. The isolated mutant ovomucoid may comprise a methionine at position 162 and an alanine at position 167 relative to SEQ ID NO: 3 when optimally aligned.

The isolated mutant ovomucoid may have reduced allergenicity relative to wild-type ovomucoid. In some cases, an isolated mutant ovomucoid has an allergenicity of about or at least 0%, 1%, 5%, 10%, 15%, 20%, 25%, 30%, 35%, 40%, 45%, 50%, 55%, 60%, 65%, 70%, 75%, 80%, 85%, 90%, or 95% relative to wild-type ovomucoid. In some cases, an isolated mutant ovomucoid has an allergenicity of up to 0%, 1%, 5%, 10%, 15%, 20%, 25%, 30%, 35%, 40%, 45%, 50%, 55%, 60%, 65%, 70%, 75%, 80%, 85%, 90%, or 95% relative to wild-type ovomucoid. In some cases, reduced allergenicity may be measured using a skin prick test, blood test, or oral food challenge.

The isolated mutant ovomucoid may have enhanced digestibility relative to wild-type ovomucoid. In some cases, an isolated mutant ovomucoid has a digestibility of about or at least 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to wild-type ovomucoid. In some cases, an isolated mutant ovomucoid has a digestibility of up to 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to wild-type ovomucoid. In some cases, enhanced digestibility may be measured as a rate of protein metabolism or rate of degradation or digestion by a protease or an acid, in vivo or in vitro.

An isolated mutant ovomucoid may have a glycosylation, acetylation, or phosphorylation pattern different from a wild-type ovomucoid. An isolated mutant ovomucoid may have a melting temperature different from a wild-type ovomucoid. An isolated mutant ovomucoid may comprise one or more amino acid insertions, deletions, or substitutions relative to a wild-type ovomucoid. In some cases, an isolated mutant ovomucoid may have about or at least 0, 1, 2, 3, 4, 5, 6, 7, 8, 9, 10, 11, 12, 13, 14, 15, 16, 17, 18, 19, 20, 25, 30, 40, 50, 60, 70, 80, 90, or 100, amino acid insertions, deletions, and/or substitutions relative to a wild-type ovomucoid. In some cases, an isolated mutant ovomucoid may have up to 0, 1, 2, 3, 4, 5, 6, 7, 8, 9, 10, 11, 12, 13, 14, 15, 16, 17, 18, 19, 20, 25, 30, 40, 50, 60, 70, 80, 90, or 100 amino acid insertions, deletions, and/or substitutions relative to a wild-type ovomucoid.

In one aspect, the present disclosure provides an egg white protein composition comprising: an isolated recombinant egg white protein or an isolated mutant ovomucoid described herein; and one or more egg white proteins. The one or more egg white proteins may be recombinantly expressed.

In one aspect, the present disclosure provides an egg white protein composition comprising two or more recombinant egg white proteins. In some cases, the two or more recombinant egg white proteins are selected from the group consisting of ovalbumin, ovotransferrin, ovomucoid, G162M F167A ovomucoid, ovoglobulin G2, ovoglobulin G3, α-ovomucin, β-ovomucin, lysozyme, ovoinhibitor, ovoglycoprotein, flavoprotein, ovomacroglobulin, ovostatin, cystatin, avidin, ovalbumin related protein X, ovalbumin related protein Y, and any combination thereof, such as from the group consisting of ovalbumin, ovotransferrin, ovomucoid, G162M F167A ovomucoid, ovoglobulin G2, ovoglobulin G3, lysozyme, ovoinhibitor, ovoglycoprotein, flavoprotein, ovomacroglobulin, ovostatin, cystatin, avidin, ovalbumin related protein X, ovalbumin related protein Y, and any combination thereof. The egg white protein composition may comprise ovalbumin. The egg white protein composition may comprise an isolated recombinant egg white protein described herein. The egg white protein composition may comprise an isolated mutant ovomucoid described herein.

The egg white proteins may have sequences derived from a single species, such as from Gallus gallus domesticus. In some cases, the single species is not Gallus gallus domesticus. The egg white proteins may have sequences derived from more than one species. In some cases, the egg white proteins have sequences derived from a bird selected from the group consisting of poultry, fowl, waterfowl, game bird, chicken, quail, turkey, duck, ostrich, goose, gull, guineafowl, pheasant, emu, and any combination thereof. In some cases, the egg white protein composition comprises three or more, four or more, or five or more or more egg white proteins. In some cases, the egg white protein composition comprises up to 5, 10, 15, or 20 egg white proteins. An egg white protein composition may comprise an isolated mutant ovomucoid disclosed herein.

In some cases, a recombinant egg white protein may comprise one or more amino acid insertions, deletions, or substitutions relative to the egg white protein in an egg white. In some cases, a recombinant egg white protein may have about or at least 0, 1, 2, 3, 4, 5, 6, 7, 8, 9, 10, 15, 20, 25, 30, 40, 50, 60, 70, 80, 90, 100, 150, 200, 250, 300, 350, 400, 450, 500, 600, 700, 800, 900, or 1000 amino acid insertions, deletions, and/or substitutions relative to the egg white protein in an egg white. In some cases, a recombinant egg white protein may have up to 0, 1, 2, 3, 4, 5, 6, 7, 8, 9, 10, 15, 20, 25, 30, 40, 50, 60, 70, 80, 90, 100, 150, 200, 250, 300, 350, 400, 450, 500, 600, 700, 800, 900, or 1000 amino acid insertions, deletions, and/or substitutions relative to the egg white protein in an egg white. For instance, a recombinant lysozyme may have an amino acid substitution (e.g., replacement of tryptophan with tyrosine) at position 62 relative to SEQ ID NO: 9 when optimally aligned.

The egg white protein composition may further comprise water. In some cases, the egg white protein composition has a percentage of water up to 5%, 10%, 15%, 20%, 25%, 30%, 35%, 40%, 45%, 50%, 55%, 60%, 65%, 70%, 75%, 80%, 85%, 90%, or 95%. In some cases, the egg white protein composition has a percentage of water within the range from 80% to 95%. In some cases, the egg white protein composition comprises at least 50%, 60%, 70%, 80%, 90%, 95%, or 99% protein by dry weight. The egg white protein composition may further comprise a food additive. In some cases, the food additive is selected from the group consisting of a sweetener, salt, carbohydrate, and any combination thereof.

The egg white protein composition may lack cholesterol. In some cases, the egg white protein composition comprises less than 10%, 5%, 4%, 3%, 2%, 1%, or 0.5% fat by dry weight. The egg white protein composition may lack fat, saturated fat, or trans fat. The egg white protein composition may lack glucose. The egg white protein composition may lack one or more egg white proteins such as ovomucoid or flavoprotein. For instance, ovomucoid is an egg white allergen, and its absence in an egg white protein composition may reduce the allergenicity of the egg white protein composition. As another example, flavoprotein may provide a yellow tinge to egg white, and its absence in an egg white protein composition may whiten the egg white protein composition or yield a brighter white color in products made with the egg white protein composition such as a meringue relative to natural egg whites. In some cases, the egg white protein composition lacks two or more, three or more, five or more, ten or more, twenty or more, or fifty or more egg white proteins. In some cases, the egg white protein composition is not an egg, egg white, or egg yolk.

In some cases, the egg white protein composition is acidic, neutral, or basic. In some cases, pH is about or at least 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some cases, pH is up to 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In some cases, the egg white protein composition may have a pH within the range from 6 to 10.

An egg white protein composition may have a foam height greater than a foam height of an egg white. In some cases, the egg white protein composition has a foam height within the range from 10 mm to 60 mm, such as from 30 mm to 60 mm. In some cases, an egg white protein composition has a foam height of about or at least 1, 5, 10, 15, 20, 25, 26, 27, 28, 29, 30, 31, 32, 33, 34, 35, 36, 37, 38, 39, 40, 41, 42, 43, 44, 45, 50, 55, or 60 mm. In some cases, an egg white protein composition has a foam height of up to 1, 5, 10, 15, 20, 25, 26, 27, 28, 29, 30, 31, 32, 33, 34, 35, 36, 37, 38, 39, 40, 41, 42, 43, 44, 45, 50, 55, or 60 mm. In some cases, an egg white protein composition has a foam height of about or at least 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. In some cases, an egg white protein composition has a foam height of up to 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white.

An egg white protein composition may have a foam seep less than a foam seep of an egg white (e.g., at 30 minutes after whipping). In some cases, the egg white protein composition may have a foam seep up to 10 mm or up to 5 mm (e.g., at 30 minutes after whipping). In some cases, an egg white protein composition has a foam seep of about or at least 0, 1, 2, 3, 4, 5, 6, 7, 8, 9, 10, 11, 12, 13, 14, 15, 20, 25, or 30 mm. In some cases, an egg white protein composition has a foam seep of up to 0, 1, 2, 3, 4, 5, 6, 7, 8, 9, 10, 11, 12, 13, 14, 15, 20, 25, or 30 mm. In some cases, foam seep is measured at 1 min, 5 min, 10 min, 15 min, 20 min, 30 min, 40 min, 50 min, 1 h, 2 h, 3 h, 4 h, 5 h, 6 h, 7 h, 8 h, 9 h, 10 h, 15 h, 20 h, 24 h, 25 h, 30 h, or more than 30 h after whipping. In some cases, an egg white protein composition has a foam seep of about or at least 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. In some cases, an egg white protein composition has a foam seep of up to 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white.

An egg white protein composition may have a foam strength greater than a foam strength of an egg white. In some cases, the egg white protein composition may have a foam strength within the range from 30 g to 100 g, such as within the range from 40 g to 100 g. In some cases, an egg white protein composition has a foam strength of about or at least 5, 10, 20, 30, 35, 40, 45, 50, 55, 60, 65, 70, 75, 80, 90, 100, 110, 120, 130, 140, 150, or 200 g. In some cases, an egg white protein composition has a foam strength of up to 5, 10, 20, 30, 35, 40, 45, 50, 55, 60, 65, 70, 75, 80, 90, 100, 110, 120, 130, 140, 150, or 200 g. In some cases, an egg white protein composition has a foam strength of about or at least 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. In some cases, an egg white protein composition has a foam strength of up to 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white.

An egg white protein composition may have a gel strength greater than a gel strength of an egg white. In some cases, the egg white protein composition may have a gel strength within the range from 100 g to 1500 g, from 500 g to 1500 g, or from 700 g to 1500 g. In some cases, an egg white protein composition has a gel strength of about or at least 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an egg white protein composition has a gel strength of up to 10, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 g. In some cases, an egg white protein composition has a gel strength of about or at least 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white. In some cases, an egg white protein composition has a gel strength of up to 0%, 1%, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% relative to an egg white.

In some cases, the egg white protein composition may have a shelf life of at least one, two, three, or six months.

An egg white protein composition may have reduced allergenicity relative to an egg white. Reduced allergenicity may be achieved, for instance, through removal of one or more egg white proteins (e.g., ovomucoid, ovalbumin, ovotransferrin, lysozyme) or removal or mutation (e.g., one or more amino acid insertions, deletions, and/or substitutions) of one or more allergenic sites or domains within an egg white protein. In some cases, an egg white protein composition has an allergenicity of about or at least 0%, 1%, 5%, 10%, 15%, 20%, 25%, 30%, 35%, 40%, 45%, 50%, 55%, 60%, 65%, 70%, 75%, 80%, 85%, 90%, or 95% relative to an egg white. In some cases, an egg white protein composition has an allergenicity of up to 0%, 1%, 5%, 10%, 15%, 20%, 25%, 30%, 35%, 40%, 45%, 50%, 55%, 60%, 65%, 70%, 75%, 80%, 85%, 90%, or 95% relative to an egg white. In some cases, reduced allergenicity may be measured using a skin prick test, blood test, or oral food challenge.

An egg white protein composition may be formulated as a liquid, solid, or powder. An egg white protein composition may be refrigerated or frozen.

In one aspect, the present disclosure provides a polynucleotide encoding an isolated recombinant egg white protein or isolated mutant ovomucoid described herein. A polynucleotide may be codon optimized. A polynucleotide may be DNA or RNA.

A polynucleotide described herein can be obtained using chemical synthesis, molecular cloning or recombinant methods, DNA or gene assembly methods, artificial gene synthesis, PCR, or any combination thereof. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable cloning or expression vector, and the cloning or expression vector in turn can be introduced into a suitable host cell for replication and amplification, as further discussed herein. Polynucleotides may be inserted into host cells by any means known in the art. Cells may be transformed by introducing an exogenous polynucleotide, for example, by direct uptake, endocytosis, transfection, F-mating, PEG-mediated protoplast fusion, Agrobacterium tumefaciens-mediated transformation, biolistic transformation, chemical transformation, or electroporation. Once introduced, the exogenous polynucleotide can be maintained within the cell as a non-integrated expression vector (such as a plasmid) or integrated into the host cell genome. The polynucleotide so amplified can be isolated from the host cell by methods well known within the art. Alternatively, nucleic acid amplification methods (e.g., PCR) allow reproduction of DNA sequences.

RNA can be obtained by using the isolated DNA in an appropriate expression vector and inserting it into a suitable host cell. When the cell replicates and the DNA is transcribed into RNA, the RNA can then be isolated using methods well known to those of skill in the art. Alternatively, RNA can be obtained by transcribing the isolated DNA, for example, by an in vitro transcription reaction using an RNA polymerase. Alternatively, RNA can be obtained using chemical synthesis.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the expression vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

A polynucleotide described herein may further encode a signal peptide. A signal peptide, also known as a signal sequence, targeting signal, localization signal, localization sequence, secretion signal, transit peptide, leader sequence, or leader peptide, may support secretion of a protein or polynucleotide. Extracellular secretion of a recombinant or heterologously expressed protein from a host cell may facilitate protein purification. For example, recovery of a recombinant protein from a cell culture supernatant may be preferable to lysing host cells to release a complex mixture of proteins including intracellular proteins of the host cell. Secretion may reduce deleterious effects that intracellular overexpression of a heterologous protein may have on a host cell such as toxicity or decreased growth rate. Secretion may allow increased protein production compared to intracellular expression in a host cell of limited volume to store the synthesized proteins. Secretory production of a protein may facilitate post-translational modification or processing (e.g., protein folding, formation of disulfide bonds, glycosylation).

A secreted protein may initially be expressed as a precursor with an N-terminal signal peptide. A signal peptide may contain a positively charged N-terminus of 1-5 residues (n-region), a central hydrophobic core of 6-16 amino acids (h-region), and a polar region of 3-7 amino acids that is a recognition site for a signal peptidase (c-region). A signal peptide may be located at the N-terminus of a preprotein that is destined for secretion out of the cell. In some cases, e.g., chicken ovalbumin and human plasminogen activator, a signal peptide is internal. A signal peptide may be 15 to 50 amino acids in length.

A signal peptide may direct the expressed precursor preprotein across the membrane of the endoplasmic reticulum. A signal peptide may be cleaved off from the rest of the protein by a signal peptidase, for example, during translocation or shortly after completion of translocation. A protein may be transported to the Golgi apparatus and secreted unless it carries a signal for retention in intracellular compartments.

A signal peptide may be located at the N-terminus of an egg white protein or mutant ovomucoid.

A signal peptide may be derived from a precursor (e.g., prepropeptide, preprotein) of a protein. Signal peptides may be derived from a precursor of a protein including, but not limited to, acid phosphatase (e.g., Pichia pastoris PHO1), albumin (e.g., chicken), alkaline extracellular protease (e.g., Yarrowia lipolytica XRP2), α-mating factor (α-MF, MAT α) (e.g., Saccharomyces cerevisiae), amylase (e.g., α-amylase, Rhizopus oryzae, Schizosaccharomyces pombe putative amylase SPCC63.02c (Amyl)), β-casein (e.g., bovine), carbohydrate binding module family 21 (CBM21)-starch binding domain, carboxypeptidase Y (e.g., Schizosaccharomyces pombe Cpyl), cellobiohydrolase I (e.g., Trichoderma reesei CBH1), dipeptidyl protease (e.g., Schizosaccharomyces pombe putative dipeptidyl protease SPBC1711.12 (Dppl)), glucoamylase (e.g., Aspergillus awamori), heat shock protein (e.g., bacterial Hsp70), hydrophobin (e.g., Trichoderma reesei HBFI, Trichoderma reesei HBFII), inulase, invertase Saccharomyces cerevisiae SUC2), killer protein or killer toxin (e.g., 128 kDa pGKL killer protein, α-subunit of the Kl killer toxin (e.g., Kluyveromyces lactic), K1 toxin KILM1, K28 pre-pro-toxin, Pichia acaciae), leucine-rich artificial signal peptide CLY-L8, lysozyme (e.g., chicken CLY), phytohemagglutinin (PHA-E) (e.g., Phaseolus vulgaris), maltose binding protein (MBP) (e.g., Escherichia coli), P-factor (e.g., Schizosaccharomyces pombe P3), Pichia pastoris Dse, Pichia pastoris Exg, Pichia pastoris Pir 1, Pichia pastoris Scw, and cell wall protein Pir4 (protein with internal repeats). A signal peptide may comprise a sequence in Table 1. In some cases, a signal peptide may be selected from the group consisting of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and any combination thereof.

In some cases, a signal peptide is about or at least 5, 10, 15, 20, 25, 30, 50, or 100 amino acids in length. In some cases, a signal peptide is up to 5, 10, 15, 20, 25, 30, 50, or 100 amino acids in length. In some cases, a signal peptide may be within the range from 5 to 50 amino acids in length or within the range from 5 to 30 amino acids in length.

A signal peptide may be modified or comprise one or more amino acid insertions, deletions, and/or substitutions, for instance, using codon optimization, directed evolution, insertion of spacers, and/or deletion mutagenesis.

A polynucleotide may further encode a signal peptidase cleavage or recognition site. A signal peptidase includes, but is not limited to, KEX2, Krpl, Enterokinase (EKT), thrombin, factor Xa (FXa), Tobacco Etch Virus (TEV), and 3C Prescission.

TABLE 1
Sequences of exemplary signal peptides

| | |
|---|---|
| SEQ ID NO: 14 | MQVKSIVNLLLACSLAVA |
| SEQ ID NO: 15 | MQFNWNIKTVASILSALTLAQA |
| SEQ ID NO: 16 | MYRNLIIATALTCGAYS ... AYVPSEPWSTLTPDASLESALKDYSQTF |
| | GIAIKSLDADKIKR |
| SEQ ID NO: 17 | MNLYLITLLFASLCSA ... ITLPKR |
| SEQ ID NO: 18 | MFEKSKFVVSFLLLLQLFCVLGVHG |
| SEQ ID NO: 19 | MQFNSVVISQLLLTLASVSMG |
| SEQ ID NO: 20 | MKSQLIFMALASLVAS ... APLEHQQQHHKHEKR |
| SEQ ID NO: 21 | MKFAISTLLIILQAAAVFA |
| SEQ ID NO: 22 | MKLLNFLLSFVTLFGLLSGSVFA |
| SEQ ID NO: 23 | MIFNLKTLAAVAISISQVSA |
| SEQ ID NO: 24 | MKISALTACAVTLAGLAIA ... APAPKPEDCTTTVQKRHQHKR |
| SEQ ID NO: 25 | MSYLKISALLSVLSVALA |
| SEQ ID NO: 26 | MLSTILNIFILLLFIQASLQ |
| SEQ ID NO: 27 | MKLSTNLILAIAAASAVVSA ... APVAPAEEAANHLHKR |
| SEQ ID NO: 28 | MFKSLCMLIGSCLLSSVLA |
| SEQ ID NO: 29 | MKLAALSTIALTILPVALA |
| SEQ ID NO: 30 | MSFSSNVPQLFLLLVLLTNIVSG |
| SEQ ID NO: 31 | MQLQYLAVLCALLLNVQS ... KNVVDFSRFGDAKISPDDTDLESRER KR |
| SEQ ID NO: 32 | MKIHSLLLWNLFFIPSILG |
| SEQ ID NO: 33 | MSTLTLLAVLLSLONSALA |
| SEQ ID NO: 34 | MINLNSFLILTVTLLSPALA ... LPKNVLEEQQAKDDLAKR |
| SEQ ID NO: 35 | MFSLAVGALLLTQAFG |
| SEQ ID NO: 36 | MKILSALLLLFTLAFA |
| SEQ ID NO: 37 | MKVSTTKFLAVFLLVRLVCA |
| SEQ ID NO: 38 | MQFGKVLFAISALAVTALG |
| SEQ ID NO: 39 | MWSLFISGLLIFYPLVLG |
| SEQ ID NO: 40 | MRNHLNDLVVLFLLLTVAAQA |
| SEQ ID NO: 41 | MFLKSLLSFASILTLCKA |
| SEQ ID NO: 42 | MFVFEPVLLAVLVASTCVTA |
| SEQ ID NO: 43 | MVSLRSIFTSSILAAGLTRAHG |
| SEQ ID NO: 44 | MFSPILSLEIILALATLQSVFA |
| SEQ ID NO: 45 | MIINHLVLTALSIALA |
| SEQ ID NO: 46 | MLALVRISTLLLLALTASA |
| SEQ ID NO: 47 | MRPVLSLLLLLASSVLA |
| SEQ ID NO: 48 | MVLIQNFLPLFAYTLFFNQRAALA |
| SEQ ID NO: 49 | MKFPVPLLFLLQLFFIIATQG |
| SEQ ID NO: 50 | MVSLTRLLITGIATALQVNA |
| SEQ ID NO: 51 | e base of the beaker to t |
| SEQ ID NO: 52 | MVLVGLLTRLVPLVLLAGTVLLLVFVVLSGG |
| SEQ ID NO: 53 | MLSILSALTLLGLSCA |
| SEQ ID NO: 54 | MRLLHISLLSIISVLTKANA |
| SEQ ID NO: 55 | MRFPSIFTAVLFAASSALA ... APVNTTTEDETAQIPAEAVIGYLDLEG |
| | DFDVAVLPFSNSTNNGLLFINTTIASIAAKEEGVSLDKR ... EAEA |
| SEQ ID NO: 56 | MFKSVVYSILAASLANA |
| SEQ ID NO: 57 | MLLQAFLFLLAGFAAKISA |
| SEQ ID NO: 58 | MASSNLLSLALFLVLLTHANS |
| SEQ ID NO: 59 | MNIFYIFLFLLSFVQG ... LEHTHRRGSLVKR |
| SEQ ID NO: 60 | MLIIVLLFLATLANS ... LDCSGDVFFGYTRGDKTDVHKSQALTAVK |
| | NIKR |
| SEQ ID NO: 61 | MESVSSLFNIFSTIMVNYKSLVLALLSVSNLKYARG ... MPTSERQQG |
| | LEER |
| SEQ ID NO: 62 | MFAFYFLTACISLKGVFG |
| SEQ ID NO: 63 | MRFSTTLATAATALFFTASQVSA |
| SEQ ID NO: 64 | MKFAYSLLLPLAGVSA ... SVINYKR |
| SEQ ID NO: 65 | MKFFAIAALFAAAAVA ... QPLEDR |
| SEQ ID NO: 66 | MQFFAVALFATSALA |
| SEQ ID NO: 67 | MKWVTFISLLFLFSSAYS ... RGVFRR |
| SEQ ID NO: 68 | MRSLLILVLCFLPLAALG |
| SEQ ID NO: 69 | MKVLILACLVALALA |
| SEQ ID NO: 70 | MFNLKTILISTLASIAVA |
| SEQ ID NO: 71 | MYRKLAVISAFLATARAQSA |

In one aspect, the disclosure provides an expression vector comprising any of the polynucleotides described herein. A polynucleotide may be located in an expression vector. An expression vector may be a construct, which is capable of delivering, and preferably expressing, one or more gene(s) or sequence(s) of interest in a host cell. Examples of expression vectors include, but are not limited to, viral vectors (e.g., adenoviruses, adeno-associated viruses, and retroviruses), naked DNA or RNA expression vectors, plasmids, cosmids, phage vectors, DNA or RNA expression vectors associated with cationic condensing agents, DNA or RNA expression vectors encapsulated in liposomes, and certain eukaryotic cells, such as producer cells. An expression vector may allow easy and efficient replication, cloning, and/or selection.

Accordingly, an expression vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. Expression vector components may generally include, but are not limited to, one or more of the following: a signal sequence; an origin of replication; one or more marker genes; and suitable transcriptional controlling elements (such as promoters, enhancers and terminator). For expression (e.g., translation), one or more translational controlling elements are also usually required, such as ribosome binding sites, translation initiation sites, internal ribosome entry site, and stop codons. The expression vector may be used to transduce, transform or infect a cell, thereby causing the cell to express nucleic acids and/or proteins other than those native to the cell. The expression vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such expression vectors are selected from among conventional vector types including insects, e.g., baculovirus expression, or yeast, fungal, bacterial or viral expression systems. Other appropriate expression vectors, of which numerous types are known in the art, can also be used for this purpose. Methods for obtaining cloning and expression vectors are well-known (see, e.g., Green and Sambrook, Molecular Cloning: A Laboratory Manual, 4th edition, Cold Spring Harbor Laboratory Press, New York (2012)).

An expression vector may further comprise a promoter. Promoters include, but are not limited to, a constitutive promoter, inducible promoter, and hybrid promoter. Promoters include, but are not limited to, acu-5, adhl+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbhl), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GALL, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2,0-galactosidase (lac4), LEU2, me1O, MET3, methanol oxidase (MOX), nmtl, NSP, pcbC, PET9, peroxin 8 (PEX8), phosphoglycerate kinase (PGK, PGK1), phol, PHO5, PHO89, phosphatidylinositol synthase (PIS1), PYK1, pyruvate kinase (pkil), RPS7, sorbitol dehydrogenase (SDH), 3-phosphoserine aminotransferase (SERI), SSA4, SV40, TEF, translation elongation factor 1 alpha (TEF1), THI11, homoserine kinase (THR1), tpi, TPS1, triose phosphate isomerase (TPI1), XRP2, and YPT1.

An expression vector may further comprise an auxotrophic marker (e.g., ade1, arg4, his4, ura3, met2). An expression vector may further comprise a selectable marker (e.g., a resistance gene). In some cases, a resistance gene may confer resistance to zeocin, ampicillin, blasticidin, kanamycin, nurseothricin, chloroamphenicol, tetracycline, triclosan, or ganciclovir. An expression vector may comprise a plasmid.

In one aspect, the present disclosure provides a host cell transformed to express one or more heterologous egg white proteins, wherein the host cell is not selected from the group consisting of Escherichia coli, Pichia pastoris, rice, Aspergillus niger, Aspergillus oryzae, Acremonium chrysogenum, Saccharomyces cerevisiae, insect, mice, corn, Pseudozyma, tobacco, zebrafish, and any combination thereof.

In one aspect, the present disclosure provides a host cell transformed to express one or more heterologous egg white proteins, wherein the one or more egg white proteins are not selected from the group consisting of ovalbumin, ovotransferrin, lysozyme, ovostatin, ovomucoid, ovoinhibitor, avidin, and any combination thereof.

In one aspect, the present disclosure provides a host cell comprising a polynucleotide or expression vector described herein. Any host cell capable of expressing heterologous DNA can be used for the purpose of isolating a protein or the polynucleotides encoding a protein. Suitable host cells include, but are not limited to, mammalian (e.g., human such as HEK or HeLa; mouse such as a 3T3 or cells derived from Swiss, BALB/c or NIH mice; hamster such as CHO; monkey such as COS), bacterial (e.g., Escherichia coli, Bacillus subtilis, Pseudomonas, Streptomyces), fungal (e.g., Saccharomyces cerevisiae, Schizosaccharomyces pombe, Kluyveromyces lactis), or insect (e.g., Drosophila melanogaster, High Five, Spodoptera frugipedera Sf9) host cells. The host cells can be transfected, e.g., by conventional means such as electroporation with at least one expression vector of the disclosure. The expression vectors containing the polynucleotides of interest can be introduced into a host cell by any of a number of appropriate means, including electroporation, chemical transformation, transfection employing calcium chloride, rubidium chloride, calcium phosphate, DEAE-dextran, or other substances; microprojectile bombardment; lipofection; PEG-mediated protoplast fusion; Agrobacterium tumefaciens-mediated transformation; biolistic transformation; and infection (e.g., where the vector is an infectious agent such as vaccinia virus). The choice of introducing expression vectors or polynucleotides will often depend on features of the host cell. The transfected or transformed host cell may then be cultured under conditions that allow expression of the protein. In some embodiments, a protein is purified from a host cell.

An expression vector may be genomically integrated. A host cell may comprise multiple copies of an expression vector. In some cases, a host cell may be selected from the group consisting of bacteria, fungi, plant, insect, mammalian, and any combination thereof. In some cases, fungi may be yeast or filamentous fungi. Yeast includes, but is not limited to, Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Yarrowia spp., and Yarrowia lipolytica. Fungi include, but are not limited to, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculosum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus. In some cases, a host cell may be selected from the group consisting of Aspergillus oryzae, Bacillus subtilis, Escherichia coli, Myceliophthora thermophila, Neurospora crassa, Pichia pastoris, and any combination thereof. A host cell may be approved as generally regarded as safe by the U.S. Food and Drug Administration. A host cell may be auxotrophic. A host cell may be glycoengineered, for instance, by having its glycosylation pathways humanized or engineered to more closely resemble another organism (e.g., a bird or chicken).

In one aspect, the present disclosure provides a cell culture comprising a host cell described herein.

In some embodiments, a polypeptide or protein is produced using in vitro or cell-free protein synthesis, for example using a cell-free translation system comprising a cell extract such as Escherichia coli cell extract, rabbit reticulocyte cell extract, wheat germ cell extract, or insect cell extract. The expressed protein may be recovered, isolated, and/or optionally purified from the cell, cell extract, or from the culture medium, by appropriate means known to one of skill in the art. For example, the proteins are isolated in soluble form following cell lysis, or extracted using known techniques, e.g., in guanidine chloride. The proteins may be further purified using any of a variety of conventional methods including, but not limited to: liquid chromatography such as normal or reversed phase, using HPLC, FPLC and the like; affinity chromatography such as with inorganic ligands or monoclonal antibodies; size exclusion chromatography; immobilized metal chelate chromatography; gel electrophoresis; and the like. One of skill in the art may select the most appropriate isolation and purification techniques. Still other suitable host cells, as well as methods for transfection, culture, amplification, screening, production, and purification are known in the art.

In one aspect, the present disclosure provides a method for making a consumable product, the method comprising substituting a portion of an egg-based ingredient with an isolated recombinant egg white protein, isolated mutant ovomucoid, or egg white protein composition described herein.

In one aspect, the present disclosure provides a method for making a consumable product, the method comprising adding an isolated recombinant egg white protein, isolated mutant ovomucoid, or egg white protein composition described herein.

In one aspect, the present disclosure provides a method of using a recombinant egg white protein as a processing agent to make a processed consumable product. In some cases, the method further comprises removing the recombinant egg white protein. The recombinant egg white protein may or may not be consumed in the processed consumable product. The processed consumable product may contain trace amounts of the recombinant egg white protein. In some cases, the processed consumable product does not contain the recombinant egg white protein.

In one aspect, the present disclosure provides a method of using an isolated recombinant egg white protein, isolated mutant ovomucoid, or egg white protein composition described herein as a processing agent to make a processed consumable product. In some cases, the method further comprises removing the isolated recombinant egg white protein, isolated mutant ovomucoid, or egg white protein composition. In some cases, the processing agent acts as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a consumable product comprising an isolated recombinant egg white protein, isolated mutant ovomucoid, or egg white protein composition described herein. A consumable product includes, but is not limited to, food product, beverage product, pharmaceutical product, and hygiene product.

In one aspect, the present disclosure provides a method of using a recombinant egg white protein as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a method of using an isolated recombinant egg white protein, isolated mutant ovomucoid, or egg white protein composition described herein as an emulsifier, binding agent, leavening agent, thickening agent, moisturizing agent, adhesive, browning agent, clarification agent, gelation agent, crystallization control agent, humectant agent, tenderizer, aeration agent, structure improvement agent, coagulation agent, coating agent, colorant, gloss agent, flavoring, freezing agent, insulation agent, mouthfeel improvement agent, pH buffer, shelf life extension agent, preservative, antimicrobial (e.g., antibacterial, antifungal, antiviral, antiparasitic), food spoilage inhibitor, malolactic fermentation inhibitor, texture improvement agent, egg replacement, or any combination thereof.

In one aspect, the present disclosure provides a method for diagnosing a food allergy, the method comprising introducing an isolated recombinant egg white protein, isolated mutant ovomucoid, or egg white protein composition described herein to a subject. In some cases, the introducing is performed using a skin prick test, blood test, or oral food challenge.

In one aspect, the present disclosure provides a method for treating a food allergy, the method comprising substituting an egg white allergen with an isolated recombinant egg white protein, isolated mutant ovomucoid, or egg white protein composition described herein or increasing a tolerance to an egg white allergen of a subject by consuming an isolated recombinant egg white protein, isolated mutant ovomucoid, or egg white protein composition described herein.

In one aspect, the present disclosure provides a method for inhibiting malolactic fermentation in a consumable product (e.g., wine), the method comprising providing an egg white lysozyme to the consumable product. The egg white lysozyme may be recombinantly expressed.

In one aspect, the present disclosure provides a method for using lysozyme (e.g., egg white lysozyme) as an antimicrobial, antiviral, preservative, or any combination thereof, for instance in food, animal feed, fruits, vegetables, pharmaceuticals, tofu, bean curd, cheese, seafood, meat, wine, sake, or beer (e.g., non-pasteurized beer). In one aspect, the present disclosure provides a method for using lysozyme (e.g., egg white lysozyme) to inhibit growth of a food spoiling organism, inhibit late-blowing (e.g., in cheese), increase shelf life, aid digestibility, treat gastrointestinal diseases, improve food safety, boost the immunity system, replace or reduce sulfites, in skin care, to cure or prevent acne or bed sores, in optical conditions, in dental conditions, in oral conditions, to treat headaches, to treat colds, to treat throat infections, or any combination thereof. The lysozyme may be recombinantly expressed.

In one aspect, the present disclosure provides a method for using ovalbumin (e.g., egg white ovalbumin) as a reference protein for immunization or biochemical studies; as a standard in the investigation of composition, physical properties, and/or structure of proteins; as a blocking agent (e.g., in immunohistochemistry or in western blots); for the detection of anti-hemoglobin monoclonal antibodies (e.g., in enzyme-linked immunosorbent assays (ELISA)); as a protein standard in molecular weight determination (e.g., by SDS-PAGE or size exclusion chromatography); in cell culture systems or in diagnostics to stabilize enzymes and hormones that would otherwise lose their functional integrity; as a protein carrier or stabilizer; or any combination thereof. The ovalbumin may be recombinantly expressed.

In one aspect, the present disclosure provides a method for using ovotransferrin (e.g., egg white ovotransferrin) as an iron-binding protein (e.g., to make iron in a bacterial culture medium nutritionally unavailable to harmful microorganisms, such as Schigella dysenteriae); as a culture media ingredient for the maturation of cells; to provide iron to cells; to detoxify culture media (e.g., by binding metal ions, such as zinc, iron, and aluminum); as a preservative; as an antiviral, antibiotic, or antimicrobial; as a lactoferrin substitute; or any combination thereof. The ovotransferrin may be recombinantly expressed.

In one aspect, the present disclosure provides a method for using avidin (e.g., egg white avidin) as a biotin binding agent; in an immunoassay; in histochemistry; in cytochemistry; in biotin purification; in chromosome visualization; in protein purification; in affinity chromatography; in affinity cytochemistry; in the study of cell surface molecular interactions; in signal amplification in immunoassay; in diagnostics; in drug delivery, targeting, or neutralization; in gene mapping; in an avidin-conjugated probe (e.g., enzymes, antigens, antibodies, lectins, hormones, nucleic acids, cells, sub-cellular organelles); or any combination thereof. The avidin may be recombinantly expressed.

In one aspect, the present disclosure provides a method for using an isolated recombinant egg white protein, isolated mutant ovomucoid, or egg white protein composition as a protein supplement. In one aspect, the present disclosure provides a method for using one or more egg white proteins as a protein supplement. In some cases, the protein supplement is formulated as a solid (e.g., powder), liquid, gel, shake, or protein bar.

Egg white protein compositions may be treated, for example, to remove glucose, preserve color, or stabilize compositions for longer storage. Glucose may be removed from a composition for long storage stability. A composition may be clarified, filtered, desugared (e.g., stabilized, glucose-free), spray dried, and/or pasteurized. Pasteurization may occur in a “hot room” maintained at a temperature of at least 130° F. (54° C.) for a minimum of seven to ten days. Pasteurization at a higher temperature may improve gel strength. Salmonella may be eliminated if the moisture content of the composition is kept at approximately 6%. Whipping ability may improve when stored in the hot room at low moisture levels. Pasteurization may occur using high temperature, short-time (HTST) pasteurization equipment. Spray drying may occur before or after pasteurization. A composition may be ultra-pasteurized. Compositions may be clarified, filtered, pasteurized, homogenized, and/or frozen at −10° to −40° F. (−23.3° to −40° C.). Compositions may be a liquid, a refrigerated liquid, frozen, or dried.

Proteins and compositions herein can be added to or mixed with one or more food additives. Food additives can add volume and/or mass to a composition. A food additive may improve functional performance and/or physical characteristics. For example, a food additive may prevent gelation or increased viscosity due to the lipid portion of the lipoproteins in the freeze-thaw cycle. An anticaking agent may be added to make a free-flowing composition. Carbohydrates can be added to increase resistance to heat damage, e.g., less protein denaturation during drying and improve stability and flowability of dried compositions. Whipping additives may be added to dried compositions (e.g., at a level of less than 0.1% by weight of the liquid prior to drying) to improve whipping ability and aeration properties. Food additives include, but are not limited to, food coloring, pH adjuster, natural flavoring, artificial flavoring, flavor enhancer, batch marker, food acid, filler, anticaking agent (e.g., sodium silicoaluminate), antigreening agent (e.g., citric acid), food stabilizer, foam stabilizer or binding agent, antioxidant, acidity regulatory, bulking agent, color retention agent, whipping agent (e.g., ester-type whipping agent, triethyl citrate, sodium lauryl sulfate), emulsifier (e.g., lecithin), humectant, thickener, pharmaceutical excipient, solid diluent, salts, nutrient, sweetener, glazing agent, preservative, vitamins, dietary elements, carbohydrates, polyol, gums, starches, flour, oil, and bran.

Food coloring includes, but is not limited to, FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), annatto, betanin, butterfly pea, caramel coloring, chlorophyllin, elderberry juice, lycopene, carmine, pandan, paprika, turmeric, curcuminoids, quinoline yellow, carmoisine, Ponceau 4R, Patent Blue V, and Green S.

pH adjuster includes, but is not limited to, Tris buffer, potassium phosphate, sodium hydroxide, potassium hydroxide, citric acid, sodium citrate, sodium bicarbonate, and hydrochloric acid.

Foam stabilizer or binding agent includes, but is not limited to, kappa carrageenan, iota carrageenan, lambda carrageenan, triethyl citrate, xanthan gum, methyl cellulose, hydroxyethyl cellulose, hydroxymethyl cellulose, and polyacrylimides.

Salts include, but are not limited, to acid salt, alkali salt, organic salt, inorganic salt, phosphates, chloride salts, sodium chloride, potassium chloride, magnesium chloride, magnesium perchlorate, calcium chloride, ammonium chloride, iron chlorides, and zinc chloride.

Nutrient includes, but is not limited to, macronutrient, macronutrient, essential nutrient, non-essential nutrient, dietary fiber, amino acid, essential fatty acids, omega-3 fatty acids, and conjugated linoleic acid.

Sweeteners include, but are not limited to, sugar substitute, artificial sweetener, acesulfame potassium, advantame, alitame, aspartame, sodium cyclamate, dulcin, glucin, neohesperidin dihydrochalcone, neotame, β-4000, saccharin, aspartame-acesulfame salt, sucralose, brazzein, curculin, glycyrrhizin, glycerol, inulin, mogroside, mabinlin, malto-oligosaccharide, mannitol, miraculin, monatin, monellin, osladin, pentadin, stevia, trilobatin, and thaumatin.

Carbohydrates include, but are not limited to, sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), monosaccharides, disaccharides, and polysaccharides (e.g., polydextrose, maltodextrin).

Polyols include, but are not limited to, xylitol, maltitol, erythritol, sorbitol, threitol, arabitol, hydrogenated starch hydrolysates, isomalt, lactitol, mannitol, and galactitol (dulcitol).

Gums include, but are not limited to, gum arabic, gellan gum, guar gum, locust bean gum, acacia gum, cellulose gum, and xanthan gum.

Vitamins include, but are not limited to, niacin, riboflavin, pantothenic acid, thiamine, folic acid, vitamin A, vitamin B6, vitamin B12, vitamin D, vitamin E, lutein, zeaxanthin, choline, inositol, and biotin.

Dietary elements include, but are not limited to, calcium, iron, magnesium, phosphorus, potassium, sodium, zinc, copper, manganese, selenium, chlorine, iodine, sulfur, cobalt, molybdenum, and bromine.

A method of making a composition may comprise drying and/or concentrating. In some cases, drying forms a dry, dehydrated, concentrated, and/or solid composition. Some non-limiting examples of drying methods include thermal drying, evaporation (e.g., by means of vacuum or air), distillation, boiling, heating in an oven, vacuum drying, spray drying, freeze drying, lyophilization, and any combination thereof. The mechanism of drying can affect the hydration and molecular structure of the composition to yield different physical properties. The composition can be dried until the composition comprises about or at least about 0.001, 0.005, 0.01, 0.05, 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 86, 87, 88, 89, 90, 91, 92, 93, 94, 95, or more than 95% solvent (e.g., water) by weight. The composition can be dried until the composition comprises up to about 0.001, 0.005, 0.01, 0.05, 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 86, 87, 88, 89, 90, 91, 92, 93, 94 or 95% solvent (e.g., water) by weight. For example, a composition can be dried via any standard drying method (e.g., 12-80 hours in an oven at 60° C., using industrial air blowers, etc.) to remove a solvent to form a dry or solid composition. In another example, a composition can be concentrated (e.g., from 80% water to 20% water).

A method of making a composition may comprise diluting and/or hydrating. In some cases, the diluting may comprise addition of a solvent. The composition can be diluted until the composition comprises about or at least about 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 86, 87, 88, 89, 90, 95, 96, 97, 98, 99, 99.5, or 99.9% water by weight. The composition can be diluted until the composition comprises up to about 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 11, 12, 13, 14, 15, 16, 17, 18, 19, 20, 25, 30, 35, 40, 45, 50, 55, 60, 65, 70, 75, 80, 85, 86, 87, 88, 89, 90, 95, 96, 97, 98, 99, 99.5, or 99.9% water by weight. For example, a composition can be diluted (e.g., from 20% water to 80% water). In another example, a dry composition can be hydrated (e.g., from a dry solid to 80% water).

### EXAMPLES

### Example 1: Recombinant Expression of Egg White Proteins in a Host Cell

A DNA plasmid or DNA oligonucleotide containing a gene sequence encoding an egg white protein is incubated with a restriction enzyme that cleaves the gene sequence at flanking restriction sequence sites. The gene sequence is isolated by agarose gel electrophoresis and gel extraction methods. The purified gene sequence is incubated with DNA ligase, DNA nucleotides as necessary, and an expression plasmid cleaved at restriction sites that leaves ends complementary to those of the isolated gene sequence, to ligate the gene downstream of a promoter (which can confer constitutive expression in a host cell, e.g., the glyceraldehyde-3-phosphate dehydrogenase promoter, or inducible expression dependent on the presence of a substance in the medium that the host cell line grows in) in the expression plasmid.

For example, a plasmid containing the gene sequence for ovalbumin flanked by the EcoRI and Sacll restriction sites respectively in the 5′->3′ direction can be cut with EcoRI and SacII restriction enzymes, isolated on an agarose gel, and ligated into a pGAPZ expression vector cut with EcoRI and Sacll.

The ligation reaction is transformed using standard methods (e.g., electroporation) into a competent cell line (e.g., Dh5alpha cell line) and plated on agar plates containing an antibiotic (e.g., Zeocin) to select for colonies of competent cells that have been transformed with the expression vector. After incubating plates for a period of time and at a temperature appropriate for growth of colonies that can be manually selected (e.g., for 16 hours at 37° C.), individual colonies are picked. The expression vector from successful transformants is isolated and purified by standard molecular biological methods (e.g., silica gel membrane or column, phenol chloroform extraction).

The expression vector is transformed into a host cell (e.g., Pichia pastoris) using standard molecular biology methods (e.g., electroporation of an electrocompetent host cell, or transformation of the host cell in the presence of polyethylene glycol or dimethyl sulfoxide). Successful transformants of the host cell by the expression vector can be selected for by spreading a solution of the transformation reaction onto a plated media (e.g., agar plate) whereby the media is appropriate for the growth of the host cell and contains a selection agent (e.g., an antibiotic corresponding to a resistance gene carried on the expression vector). The plated media is incubated for an appropriate amount of time and at an appropriate temperature until individual colonies of the host cell can be isolated from the plate (e.g., 30° C. for one week). The resultant clones are individually isolated and plated separately on fresh selection plates and incubated again. Individual colonies from these plates are used to inoculate individual culture vessels containing appropriate growth medium for the host cell with the same selection agent as used in the initial round. After an appropriate amount of time (e.g., overnight at 30° C. in a shaker flask), successful transformation of the host cell with the expression vector can be determined in each culture vessel by the presence of protein coded by the gene sequence versus a control vessel that is inoculated with a colony from a negative control plate as determined by standard molecular biology methods (e.g., Western blot). Colonies from selection plates corresponding to culture vessels showing protein expression can be used to inoculate vessels containing media appropriate for the host cell to promote growth of the host cell and secretion of the protein into the media. Alternatively, colonies from plates corresponding to culture vessels showing protein expression can be stored for later use (e.g., at −80° C. in a DMSO solution).

### Example 2: Choice of a Host Cell and Comparison of Recombinant Proteins to Native Proteins

DNA sequences encoding a protein component of egg white can be synthesized and cloned into an expression vectors for expression in a host cell (e.g., yeast, filamentous fungi).

For example, the yeast strain Pichia pastoris may be a suitable a host species for the recombinant vectors, due to its efficiency in recombinant expression and protein secretion, particularly for proteins with disulfide bonds. Pichia pastoris is grown in glycerol-containing BMGY media for two days and switched to methanol-containing BMMY media to induce recombinant protein production and grown for two days to a week in a flask with shaking at 30° C.

Recombinant proteins can be compared to native protein, for instance through protein conformation, activity, acetylation pattern, phosphorylation pattern, glycosylation pattern, gelation properties, or other functional properties.

The above scheme is to be optimized in order that protein yields/(L of culture)/day can be increased to achieve optimal output capacity and production cost.

### Example 3: Purification of Recombinant Proteins

Purified recombinant proteins can be obtained from cultures of transformed cell lines.

The desired yield of the protein (e.g., in grams) can be obtained with appropriate sized fermentation vessels and culturing time. Secreted recombinant proteins can be purified from the culture supernatant (e.g., by spinning down culture media in a centrifuge). For example, host cells are removed from the cell culture supernatant by centrifugation. The proteins in the supernatant are then purified by hollow fiber diafiltration. In a second example, proteins may be purified with a mild salt extraction, followed by centrifugation.

Alternative vessel designs can allow continuous circulation of media, and filtration in a separate vessel to collect protein secretions without interrupting cell growth (e.g., a hollow fiber bioreactor).

Purified recombinant proteins can be dialyzed with an aqueous buffer of appropriate pH that is suitable for gelation upon heating to obtain a wet egg white protein composition or for downstream lyophilization (e.g., spray drying) to obtain a powdered egg white protein composition.

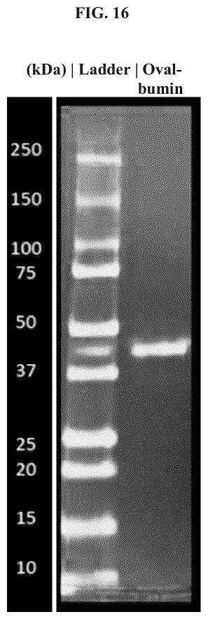

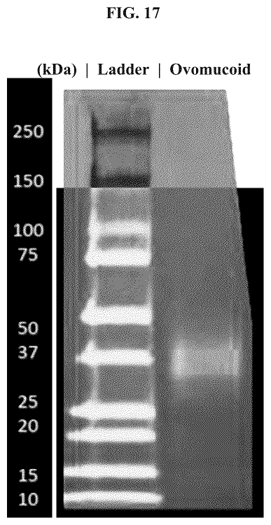

Purified recombinant proteins can be characterized by Coomassie stained 4-20% sodium dodecyl sulfate polyacrylamide gel electrophoresis (SDS-PAGE), as shown for example in FIG. 16 and FIG. 17.

### Example 4: Combination of Purified Recombinant Proteins into an Egg White Protein Composition

The construction of recombinant vectors and purification of their protein products in a transformed cell line can be carried out separately for each egg white protein constituent that is to be included in the final formulation for the egg white protein composition. Desired combinations and amounts of these purified proteins in the composition can be added into one volume to achieve specific final concentrations of each protein. For example, one formulation could include constituent recombinant proteins added together in final concentrations that match their corresponding concentration in animal derived egg whites.

An egg white protein composition can be stored and refrigerated as a wet egg white protein composition. Alternatively, an egg white protein composition may first be heated to induce gelation (e.g., at a temperature sufficient to induce denaturation of the most unstable constituent protein), and then stored as a refrigerated product or lyophilized for a powdered egg white protein composition. Alternatively, egg white proteins may be combined in lyophilized form to form an egg white protein composition and then dissolved in solution. The concentration and components of salts and food additives in the solution may vary depending on formulation.

Varying amounts of protein components can be mixed together in proportions matching those found in animal derived egg whites, and spray dried to be packaged as dried egg white protein, which can be reconstituted with the addition of water. Alternatively, the egg protein mix can be subjected to heating, and consequent gelation of the substance can be packaged as a refrigerated ready-made egg white. Factors such as protein mix composition, pH, percentage of water, rate of heating can be varied to produce variations in consistency and palatability.

### Example 5: Isolation of Egg White from an Egg

An egg is brought to room temperature (e.g., 25° C.) by leaving the uncracked egg outside for at least 30 minutes. The egg white is separated from the yolk.

### Example 6: Foaming and Foam Stability of Whipped Egg White or Egg White Protein Composition

Egg white or egg white protein composition (10 mL) is added to a 50 mL Pyrex beaker. The beaker is placed on a Dremel rotary tool work station stand with a Dremel 3000 variable speed rotary tool mounted to it with a 0.5 inch steel brush attachment. The stand is adjusted such that when the Dremel is lowered into the beaker, the brush barely touches the bottom of the beaker and is fully submerged in the egg white. From this submerged starting position, the Dremel tool is turned on to speed setting 3. The steel brush whips the egg white for 1 minute. After 1 minute, the Dremel tool is turned off, the attachment is raised, and the beaker is removed.

Using calipers with at least 0.5 mm accuracy, the foam height is measured as the distance from the liquid-foam interface to the foam-air interface. If no liquid is visible, the foam height is measured as the distanced from the base of the beaker to the top of the foam-air interface.

To measure seeping and/or foam stability, the amount of liquid is measured 10 minutes after shutting off the Dremel tool using the calipers as the distance from the base of the beaker to the liquid-foam interface. The amount of liquid is measured again 30 minutes after shutting off the Dremel tool.

The foam height of the whipped egg white is approximately 30 mm.

The foam height of the whipped egg white protein composition may be approximately 36.25 mm.

The foam seep of the whipped egg white is approximately 2.5 mm after 10 min and 8 mm after 30 min.

The foam seep of the whipped egg white protein composition may be approximately 0 mm after 10 min and 0.5 mm after 30 min.

### Example 7: Foam Strength and Texture Analysis of Whipped Egg White or Egg White Protein Composition

A recrystallization dish is filled with at least 40 mm of whipped egg white foam or whipped egg white protein composition foam. A trigger value of 3 g is set along with a deformation of 20 mm using a Brookfield TA-MP probe on a Brookfield CT3 Texture Analyzer. The zero height is set to be less than 5 mm above the surface of the egg white foam or egg white protein composition foam. Testing is performed on the Normal setting at least in triplicate and immediately after whipping egg white or egg white protein composition. The whipped egg white or whipped egg white protein composition is tested within 30 minutes of whipping to minimize error due to seepage. Different surface areas of the foam are chosen between tests that are not previously tested so that any collapsed foam bubbles from previous testing do not introduce error in subsequent tests. The initial noise prior to the trigger value is taken into account for error measurements. The difference in peak load between the 3 runs performed per sample is also recorded.

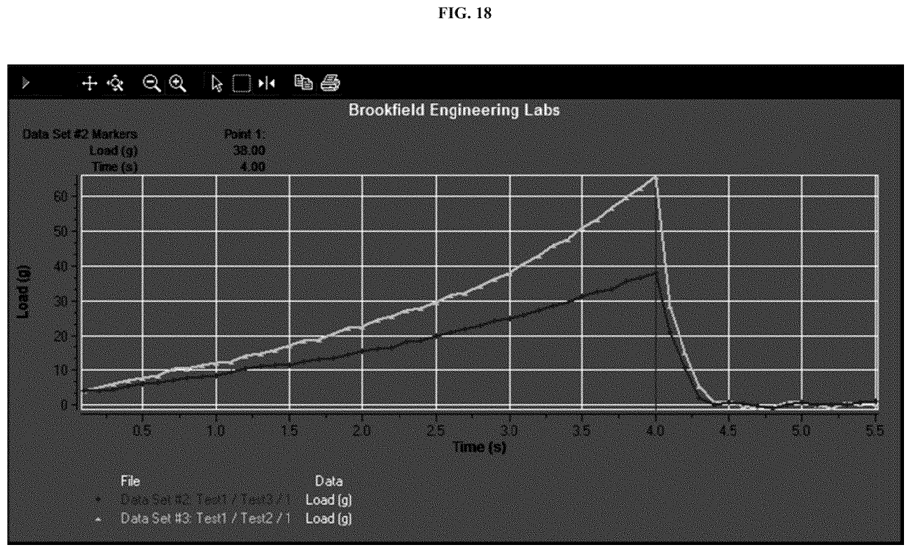

The foam strength of the whipped egg white is approximately 38 g, as shown for example in FIG. 18.

The foam strength of the whipped egg white protein composition is approximately 62 g, as shown for example in FIG. 18.

### Example 8: Gel Strength of Cooked Egg White or Egg White Protein Composition

Egg white or egg white protein composition (10 mL) is added to a 50 mL conical falcon tube. The tube is boiled in a water bath at 95° C. for 9 minutes. The tube is removed and allowed to cool to room temperature. The tube is placed in a tube holder on a Brookfield CT3 Texture Analyzer and a Brookfield TA-10 probe is lowered to ˜2 mm above the surface of the cooked egg white or egg white protein composition. The analyzer is run with a trigger value of 3 g and a deformation of 2 mm. The gel strength of the cooked egg white or egg white protein composition is measured as the peak hardness seen over the 2 mm deformation on the first run.

The gel strength of the cooked egg white is approximately 500 to 700 g.

The gel strength of the cooked egg white protein composition is approximately 150 to 500 g.

### Example 9: Emulsifying Capacity and Emulsion Stability of Egg White or Egg White Protein Composition

To evaluate the emulsifying capacity, oil is added gradually to a solution containing egg white or egg white protein composition. The amount of oil required for transition from an oil in water to a water in oil emulsion is determined.

To evaluate the stability of the emulsion, the amount of oil or water separated from the emulsion is determined after leaving the emulsion under certain conditions.

### Example 10: Angel Food Cake with Egg White or Egg White Protein Composition

Egg white or egg white protein composition (30 g) is brought to room temperature and placed in the mixing bowl of a KitchenAid stand mixer with a whipping attachment. The egg white or egg white protein composition is beaten on speed 5 until soft peaks form. Finely granulated sugar (18 g) is slowly added. The mixture is beaten on speed 8 until stiff peaks form. In a separate bowl, sugar (18 g) and flour (24 g) are sifted together. The sugar and flour mixture is folded into the mixture on speed 2. The batter is spooned into a round angel food cake pan and baked in a preheated oven at 200° F. for 30 minutes. After the pan is removed from the oven, it is immediately inverted and allowed to cool before the cake is removed from the pan.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An animal-free egg white composition comprising a recombinantly-produced ovalbumin;
wherein the recombinant ovalbumin provides an equivalent or an improvement in a functional property compared to an otherwise similar animal-free egg white composition comprising animal-derived egg white, wherein the property is selected from the group consisting of: gelling, foam capacity, foam stability, whipping, fluffing, binding, aeration, coating, film forming, emulsification, browning, thickening, texturizing, humectant, and clarification.

2. The animal-free egg white composition of claim 1, further comprising two or more additional recombinantly-produced protein.

3. The animal-free egg white composition of claim 2, wherein the two or more additional recombinantly-produced proteins are selected from: recombinant ovomucoid; recombinant lysozyme; and recombinant ovotransferrin.

4. The animal-free egg white composition of claim 1, further comprising one or more additional recombinantly-produced proteins selected from recombinant ovomucoid; recombinant lysozyme; and recombinant ovotransferrin.

5. The animal-free egg white composition of claim 1, wherein the composition is dried.

6. The animal-free egg white composition of claim 5, wherein the composition is spray-dried.

7. The animal-free egg white composition of claim 1, wherein the composition is heat-treated.

8. The animal-free egg white composition of claim 1, wherein the composition is gelled.

9. The animal-free egg white composition of claim 1, wherein the composition is liquid.

10. The animal-free egg white composition of claim 1, wherein the recombinantly-produced proteins are produced in a yeast cell.

11. The animal-free egg white composition of claim 10, wherein the yeast cell is a Pichia pastoris cell.

12. The animal-free egg white composition of claim 1, wherein the recombinantly-produced ovalbumin is secreted from a Pichia pastoris cell.

13. The animal-free egg white composition of claim 10, wherein the yeast cell is selected from: a Trichoderma reesei and a Aspergillus niger.

14. The animal-free egg white composition of claim 1, wherein the composition is packaged as a ready-made egg white.

15. The animal-free egg white composition of claim 1, wherein the glycosylation pattern of one or more recombinant proteins is different from animal-derived proteins.

16. An animal-free egg white composition comprising a recombinantly-produced ovalbumin;
wherein the animal-free egg white composition comprising recombinantly-produced ovalbumin resembles an animal-derived egg white in one or more of consistency, taste, functional properties, and appearance,
wherein the functional properties are selected from the group consisting of: gelling, foam capacity, foam stability, whipping, fluffing, binding, aeration, coating, film forming, emulsification, browning, thickening, texturizing, humectant, and clarification.

17. The animal-free egg white composition of claim 16, further comprising two or more additional recombinantly-produced protein.

18. The animal-free egg white composition of claim 17, wherein the two or more additional recombinantly-produced proteins are selected from: recombinant ovomucoid; recombinant lysozyme; and recombinant ovotransferrin.

19. The animal-free egg white composition of claim 16, further comprising one or more additional recombinantly-produced proteins selected from recombinant ovomucoid; recombinant lysozyme; and recombinant ovotransferrin.

20. The animal-free egg white composition of claim 16, wherein the composition is dried.

21. The animal-free egg white composition of claim 20, wherein the composition is spray-dried.

22. The animal-free egg white composition of claim 16, wherein the composition is heat-treated.

23. The animal-free egg white composition of claim 16, wherein the composition is gelled.

24. The animal-free egg white composition of claim 16, wherein the composition is liquid.

25. The animal-free egg white composition of claim 16, wherein the recombinantly-produced proteins are produced in a yeast cell.

26. The animal-free egg white composition of claim 25, wherein the yeast cell is a Pichia pastoris cell.

27. The animal-free egg white composition of claim 16, wherein the recombinantly-produced ovalbumin is secreted from a Pichia pastoris cell.

28. The animal-free egg white composition of claim 25, wherein the yeast cell is selected from: a Trichoderma reesei and a Aspergillus niger.

29. The animal-free egg white composition of claim 16, wherein the composition is packaged as a ready-made egg white.

30. The animal-free egg white composition of claim 16, wherein the glycosylation pattern of one or more recombinant proteins is different from animal-derived proteins.
